(19) United States
(12) Patent Application Publication
(10) Pub. No.: US 2026/0337285 A1
(43) Pub. Date: Oct. 29, 2026

(54) ANTI-AMYLIN RECEPTOR ANTIBODIES, ANTIBODY FRAGMENTS, AND NANOBODIES

(71) Applicant: AstralBio, Inc., Boston, MA (US)

(72) Inventors: Cody Allen Moore, San Diego, CA (US); Hongyu Zhang, San Diego, CA (US); Alexander Tomoaki Taguchi, San Diego, CA (US); Arjan Hada, San Diego, CA (US); Tom Sih-Yuan Hsu, San Diego, CA (US); Courtney Wood, San Diego, CA (US); Cory Schwartz, San Diego, CA (US); Martin Brenner, San Diego, CA (US); Patrick Crutcher, Boston, MA (US); Justin DiMartino, Boston, MA (US)

(21) Appl. No.: 19/654,778

(22) Filed: Apr. 22, 2026

Related U.S. Application Data
(60) Provisional application No. 63/792,740, filed on Apr. 22, 2025.

(51) Int. Cl. C07K 16/28; A61K 39/00; A61P 3/04; C07K 14/72

(52) U.S. Cl. CPC C07K 16/2869; A61P 3/04; C07K 14/72; A61K 2039/505; C07K 2317/569; C07K 2317/622; C07K 2317/92

(57) ABSTRACT

Provided herein are anti-amylin receptor antibody, antigen-binding fragments thereof, or nanobody have specific complementarity determining regions (CDRs) for the heavy and light chains, heavy and light chains, antigen-binding fragments thereof, polynucleotides that encode the same, vectors, and host cells, and methods for treating a metabolic disorder, type 2 diabetes, obesity, an elevated triglyceride level, lipodystrophy, liver inflammation, fatty liver disease, hypercholesterolemia, an elevated liver enzyme, nonalcoholic steatohepatitis (NASH), a cardiovascular disease, cardiomyopathy, high blood pressure, and/or heart failure with the anti-amylin receptor antibody, antigen-binding fragments thereof, or nanobody disclosed herein.

### CROSS-REFERENCE TO RELATED APPLICATIONS

This application claims priority to U.S. Provisional Application Ser. No. 63/792,740, filed Apr. 22, 2025, the entire contents of which are incorporated herein by reference.

### TECHNICAL FIELD

This document relates to materials and methods for treating obesity, and particularly to the use of anti-amylin receptor antibodies, antigen-binding fragments thereof, and/or nanobodies, to reduce or eliminate weight, and to treat obesity and related diseases.

### REFERENCE TO ELECTRONIC SEQUENCE LISTING

The application contains a Sequence Listing which has been submitted electronically in .XML format and is hereby incorporated by reference in its entirety. Said .XML copy, created on Apr. 21, 2026, is named “IBIO-1047.xml” and is 159,771 bytes in size. The sequence listing contained in this .XML file is part of the specification and is hereby incorporated by reference herein in its entirety.

### BACKGROUND

Without limiting the scope of the invention, its background is described in connection with amylin receptor antibodies.

Pramilintide is a peptide that has been FDA approved for diabetes treatment (since 2005) and has shown efficacy for weight loss. Pramilintide is a synthetic amylin analog of amylin. Amylin is a natural, pancreatic islet peptide that is normally secreted with insulin in response to meals. Amylin has several beneficial effects on glucose homeostasis: suppression of glucagon secretion, delaying of gastric emptying, and promotion of satiety. Pramilintide is dosed before meals by subcutaneous injection, often in conjunction with insulin. However, a limitation with the use of Pramilintide is that it requires multiple subcutaneous doses per day.

To address the issue above, one such formulation is taught in U.S. Publication No. 20240122847, filed by Bhami, et al, and is entitled “Oral Polypeptide Formulations”. These applicants are said to teach compositions that include a polypeptide formulation, e.g., truffle, tablet, globule, or candy formulation suitable for buccal/sublingual administration, where the formulation is an amorphous polypeptide composition or a crystallized polypeptide composition and a pharmaceutically acceptable carrier.

Despite these advances, a need remains for novel composition and methods for reducing weight loss by addressing glucose homeostasis, namely: suppression of glucagon secretion, delaying of gastric emptying, and promotion of satiety. Novel compositions and methods are needed that do not require daily subcutaneous dosing or the formation of amorphous polypeptide or crystallized polypeptide compositions.

### SUMMARY

As embodied and broadly described herein, an aspect of the present disclosure relates to an anti-amylin receptor antibody, antigen-binding fragments thereof, or nanobody, wherein the antibody, antigen-binding fragments thereof, or nanobody comprises: a heavy chain variable domain (VH) complementarity determining region (CDR) 1, VH CDR2, and VH CDR3 comprising an amino acid sequence of SEQ ID NOS: 8, 10, 12; 42, 44, 46; 76, 78, 80; 110, 112, 114; 144, 146, 148; 178, 180, 182; or 315, 316, 317, respectively; and a light chain variable domain (VL) CDR1, VL CDR2, VL CDR3 comprising an amino acid sequence of SEQ ID NOs: 22, 24, 26; 56, 58, 60; 90, 92, 94; 124, 126, 128; 158, 160, 162; 192, 194, 196; or 318, 319, 320, respectively; or a VHH CDR1, CDR2, CDR3 comprising an amino acid sequence of SEQ ID NOs: 210, 212, 214; 228, 230, 232; 246, 248, 250; 264, 266, 268; 282, 284, 286; or 300, 302, 304. In one aspect, the antibody comprises: a VH comprising an amino acid having at least 90, 91, 92, 93, 94, 95, 96, 97, 98, 99, or 100% sequence identity to SEQ ID NOS: 3, 37, 71, 105, 139, 173, or 305 and a VL comprising the amino acid sequence of any one of the following SEQ ID NOs: an amino acid having at least 90, 91, 92, 93, 94, 95, 96, 97, 98, 99, or 100% sequence identity to SEQ ID NOS: 5, 39, 73, 107, 141, 175, or 307; a VHH comprising an amino acid having at least 90, 91, 92, 93, 94, 95, 96, 97, 98, 99, or 100% sequence identity to SEQ ID NOS: 210, 228, 246, 264, 282, or 300; or is an ScFv having an amino acid having at least 90, 91, 92, 93, 94, 95, 96, 97, 98, 99, or 100% sequence identity to SEQ ID NO: 1, 35, 69, 103, 137, or 171. In another aspect, the antibody is a monoclonal antibody. In another aspect, the antibody is a full-length antibody. In another aspect, the antibody is an antibody fragment selected from Fab, Fab′, F(ab′)2, Fv, single-chain variable fragment (scFv), or VHH. In another aspect, the antibody or nanobody is fused to an Fc domain of any one of the following: human IgG1, human IgG2, human IgG3, and human IgG4.

As embodied and broadly described herein, an aspect of the present disclosure relates to a method of treating a disease in a subject in need thereof, comprising administering to the subject a therapeutically effective amount of the antibody or nanobody described hereinabove. In one aspect, the disease is obesity. In another aspect, the disease is selected from at least one of: a metabolic disorder, type 2 diabetes, an elevated triglyceride level, lipodystrophy, liver inflammation, fatty liver disease, hypercholesterolemia, an elevated liver enzyme, nonalcoholic steatohepatitis (NASH), a cardiovascular disease, cardiomyopathy, high blood pressure, or heart failure. In another aspect, the subject is human.

As embodied and broadly described herein, an aspect of the present disclosure relates to a scFv anti-amylin receptor antibody wherein the antibody comprises an scFv1 and an scFv2 binding site in tandem on each antibody arm and wherein said scFv1 and scFv2 are linked by a linker, optionally a flexible linker. In one aspect, the antibody has a total of four scFv binding sites in a single scFv-Fc formatted antibody. In another aspect, the scFv1 of each antibody arm comprises a first heavy chain variable domain (VH1) and a first light chain variable domain (VL1); and wherein the scFv2 of each antibody arm comprises a first heavy chain variable domain (VH2) and a first light chain variable domain (VL2). In another aspect, the antibody or antigen-binding fragment thereof comprises: at least 90, 91, 92, 93, 94, 95, 96, 97, 98, 99, or 100% sequence identity to SEQ ID NOS: 1, 35, 69, 103, 137, or 171. In another aspect, the linker comprises the following amino acid sequence: SGG, GGG, GGS, SSG, SSS, or multiples or combinations thereof.

As embodied and broadly described herein, an aspect of the present disclosure relates to an method of treating a disease in a subject in need thereof, comprising administering to the subject a therapeutically effective amount of the antibody described hereinabove. In one aspect, the disease is obesity. In another aspect, the disease is selected from at least one of: a metabolic disorder, type 2 diabetes, obesity, an elevated triglyceride level, lipodystrophy, liver inflammation, fatty liver disease, hypercholesterolemia, an elevated liver enzyme, nonalcoholic steatohepatitis (NASH), a cardiovascular disease, cardiomyopathy, high blood pressure, or heart failure. In another aspect, the subject is human.

As embodied and broadly described herein, an aspect of the present disclosure relates to a nucleic acid that expressed an anti-amylin receptor antibody, antigen-binding fragments thereof, or nanobody, wherein the antibody, antigen-binding antigen-binding thereof, or nanobody comprising: a heavy chain variable domain encoded by a polynucleotide having at least 95, 96, 97, 98, 99, or 100% sequence identity to SEQ ID NOS: 8, 10, 12; 42, 44, 46; 76, 78, 80; 110, 112, 114; 144, 146, 148; or 178, 180, 182; or 310; and a light chain variable domain encoded by a polynucleotide having at least 95, 96, 97, 98, 99, or 100% sequence identity to SEQ ID NOS: 22, 24, 26; 56, 58, 60; 90, 92, 94; 124, 126, 128; 158, 160, 162; or 192, 194, 196; or 312; or a VHH encoded by a polynucleotide having at least 95, 96, 97, 98, 99, or 100% sequence identity to SEQ ID NOS: 212, 230, 248, 266, 284, or 302. In one aspect, the antibody is a monoclonal, bispecific, multivalent, multi-specific, diabody, chimeric, scFv antibody, or antigen-binding fragments thereof. In another aspect, an antibody binding domain is fused to an Fc domain of any one of the following: human IgG1, human IgG2, human IgG3, and human IgG4. In another aspect, the nucleic acid sequence is optimized for expression in a bacterial, fungal, mammalian, insect, or plant cell. In another aspect, the antibody or antigen binding fragment is encoded by a polynucleotide having at least 95, 96, 97, 98, 99, or 100% sequence identity to SEQ ID NOS; 2, 36, 70, 104, 138, 172, or 313 or 212, 230, 248, 266, 284, 302, or 314.

As embodied and broadly described herein, an aspect of the present disclosure relates to a vector comprising the nucleic acid described hereinabove. As embodied and broadly described herein, an aspect of the present disclosure relates to a host cell comprising nucleic acid the vector described hereinabove.

As embodied and broadly described herein, an aspect of the present disclosure relates to an antigenic peptide an amino acid having at least 90, 91, 92, 93, 94, 95, 96, 97, 98, 99, or 100% sequence identity to SEQ ID NOS: 206, 308, or 309. In one aspect, the peptide is encoded by a polynucleotide having at least 95%, 96%, 97%, 98%, 99%, or 100% sequence identity to SEQ ID NOS: 308, 311, or 313.

### BRIEF DESCRIPTION OF THE DRAWINGS

For a more complete understanding of the features and advantages of the present invention, reference is now made to the detailed description of the invention along with the accompanying figures and in which:

FIG. 1 illustrates the design of the engineered antigens designed for Amylin3 Receptor.

FIGS. 2A to 2C show the validation using Soluble Calcitonin Receptor Validated by Salmon Calcitonin Binding. FIG. 2A shows the binding to the Extracellular Domain (ECD) Binding to Salmon Calcitonin, FIG. 2B shows the binding to the Transmembrane Domain (TMD) Binding to Salmon Calcitonin, and FIG. 2C shows the binding to the ECD+TMD Binding to Salmon Calcitonin.

FIGS. 3A to 3C show the validation which shows that the CTR-RAMP3 (Amylin3) Engineered Antigen Binds Anti-CTR. FIG. 3A binding to the CTR extracellular domain (ECD), FIG. 3B binding to RAMP3, and FIG. 3C binding to Amylin 3.

FIG. 4 shows the results from Engineered Antigen Steered Strategies Yield Receptor Specific Cell Binders using engineered antigen steered strategies.

FIG. 5 shows the results from Naïve scFv and VHH Libraries Yield Amylin3 Receptor Specific Cell Binders.

FIGS. 6A and 6B show the effect of test articles on acute food intake, measured at 3 hours (FIG. 6A) and 6 hours (FIG. 6B) post-feeding. Dosing was performed 4 hours prior to feeding.

### DETAILED DESCRIPTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

It should be understood that, unless clearly indicated, in any method described or disclosed herein that includes more than one act, the order of the acts is not necessarily limited to the order in which the acts of the method are recited, but the disclosure encompasses exemplary embodiments in which the order of the acts is so limited.

All publications, patents, and patent applications mentioned in this specification are herein incorporated by reference to the same extent as if each individual publication, patent, or patent application was specifically and individually indicated to be incorporated by reference.

Amylin, or islet amyloid polypeptide (IAPP), is a 37-amino acid peptide hormone in its mature form. Amylin is co-secreted with insulin from the pancreatic β-cells in the ratio of approximately 100:1 (insulin:amylin) and plays a role in glycemic regulation by slowing gastric emptying and promoting satiety, thereby preventing post-prandial spikes in blood glucose levels.

Amylin is processed from an initial 89 amino acid coding sequence in the pancreatic beta cells (β-cells) into a 67 amino acid, 7404 Dalton pro-peptide. The pro-peptide undergoes post-translational modifications including protease cleavage to produce amylin. The 67 amino acid peptide follow a 22 amino acid signal peptide that is rapidly cleaved after translation of the 89 amino acid coding sequence. The human sequence (from N-terminus to C-terminus) is: (MGILKLQVFLIVLSVALNHLKA) (SEQ ID NO: 303), which after cleavage has the sequence: TPIESHQVEKR\hat{ }KCNTATCATQRLANFLVHSSNNFGAILSSTNVGSNTYG\hat{ }KR\hat{ }NAVE VLKREPLNYLPL (SEQ ID NO: 321). UniProt P10997, RefSeq NM_000415.

The 22 amino acid signal peptide is removed during translation of the protein and transported into the endoplasmic reticulum. Inside the endoplasmic reticulum, a disulfide bond is formed between cysteine residues numbers 2 and 7. Next, in the secretory pathway, the amylin precursor undergoes additional proteolysis and posttranslational modification, including: 11 amino acids are removed from the N-terminus by the enzyme proprotein convertase 2 (PC2), 16 amino acids are removed from the C-terminus of the amylin precursor molecule by proprotein convertase 1/3 (PC1/3), and at the C-terminus, Carboxypeptidase E removes the terminal lysine and arginine residues. The terminal glycine amino acid allows the enzyme peptidylglycine alpha-amidating monooxygenase (PAM) to add an amine group. After this the transformation from the precursor amylin protein to the biologically active amylin is complete (Amylin/IAPP sequence: KCNTATCATQRLANFLVHSSNNFGAILSSTNVGSNTY (SEQ ID NO: 322).

The amylin receptors (AMYRs) are heterodimers of the calcitonin receptor and receptor activity modifying proteins (RAMPs). Amylin binds with high affinity and consist of AMY1, AMY2, and AMY3. RAMP 1 is a protein that in humans is encoded by the RAMP1 gene, RefSeq: NM_001308353 or NM_005855, UniProt: 060894. RAMPs are type I transmembrane proteins with an extracellular N terminus and a cytoplasmic C terminus. RAMPs are required to transport calcitonin-receptor-like receptor (CALCRL) to the plasma membrane. The RAMP1 protein interacts with the calcitonin receptor (CT) protein, where heteromerization of RAMP1 with CT converts CT from a calcitonin receptor to the amylin receptor AMY1.

As used herein, the term “antibody” refers to an intact antibody or an antigen-binding fragment that binds specifically to a target antigen. Antigen-binding fragments are produced by recombinant DNA techniques, or by enzymatic or chemical cleavage of intact antibodies. Antigen-binding fragments include Fab, Fab′, F(ab′)2, Fv, and single-chain variable fragment (scFv) antibodies. An antibody substantially inhibits adhesion of a receptor to a counterreceptor when an excess of antibody reduces the quantity of receptor bound to counterreceptor by at least about 20%, 40%, 60% or 80%, and more usually greater than about 85% (as measured in an in vitro competitive binding assay). The term “antibody” is used in the broadest sense, and specifically covers monoclonal antibodies (including full-length antibodies or other bivalent, Fc-region containing antibodies such as bivalent scFv Fc-fusion antibodies), polyclonal antibodies, multispecific antibodies (e.g., bispecific antibodies), and antigen-binding fragments (e.g., Fab, Fab′, F(ab′)₂, Fv, scFv) so long as they exhibit the desired biological activity. Antibodies (Abs) and immunoglobulins (Igs) are glycoproteins having the same structural characteristics. The present invention includes monoclonal antibodies (and antigen-binding fragments thereof) that are completely recombinant, in other words, where the complementarity determining regions (CDRs) are genetically spliced into a human antibody backbone, often referred to as veneering an antibody. Thus, in certain aspects, the monoclonal antibody is a fully synthesized antibody. In certain embodiments, the monoclonal antibodies (and antigen-binding fragments thereof) can be made in bacterial or eukaryotic cells, including plant cells.

As used herein, the term “antibody fragment” refers to a portion of a full-length antibody, generally the antigen-binding or variable region, and include Fab, Fab′, F(ab′)₂, Fv, and scFv fragments. Papain digestion of antibodies produces two identical antigen-binding fragments, called the Fab fragment, each with a single antigen-binding site, and a residual “Fc” fragment, so-called for its ability to crystallize readily. Pepsin treatment yields an F(ab′)2 fragment that has two antigen-binding fragments which are capable of cross-linking antigen, and a residual other fragment (which is termed pFc′). As used herein, “functional fragment” with respect to antibodies, refers to Fv, F(ab) and F(ab′)2 fragments.

As used herein, the “Fv” fragment is the minimum antibody fragment that contains a complete antigen recognition and binding site. This region consists of a dimer of one heavy and one light chain variable domain in a tight, non-covalent association (VH-VL dimer). It is in this configuration that the three CDRs of each variable domain interact to define an antigen-binding site on the surface of the VH-VL dimer. Collectively, the six CDRs confer antigen-binding specificity to the antibody. However, even a single variable domain (or half of an Fv comprising only three CDRs specific for an antigen) has the ability to recognize and bind antigen, although at a lower affinity than the entire binding site.

The Fab fragment, also designated as F(ab), also contains the constant domain of the light chain and the first constant domain (CH1) of the heavy chain. Fab′ fragments differ from Fab fragments by the addition of a few residues at the carboxyl terminus of the heavy chain CH1 domain including one or more cysteines from the antibody hinge region. Fab′-SH is the designation herein for Fab′ in which the cysteine residue(s) of the constant domains have a free thiol group. F(ab′) fragments are produced by cleavage of the disulfide bond at the hinge cysteines of the F(ab′)2 pepsin digestion product. Additional chemical couplings of antibody fragments are known to those of ordinary skill in the art.

Antibody fragments of the disclosure have amylin receptor antigen binding specificity. Antibody fragments include antigen-binding fragments (Fab), variable fragments (Fv) containing VH and VL sequences, single chain variable fragments (scFv) containing VH and VL sequences linked together in one chain, single chain antibody fragments (scAb) or other antibody variable region fragments, such as retaining antigen binding specificity.

The present disclosure also includes “nanobody” or “nanobodies”, which have the structure: VHH, that is, they have only one variable domain, unlike conventional antibodies with both light and heavy chains. Nanobodies are well-known, including descriptions in patent applications referenced as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N.V. and Ablynx N.V.; WO 01/90190 by the National Research Council of Canada; WO 03/025020 (=EP 1 433 793) by the Institute of Antibodies; as well as WO 04/041867, WO 04/041862, WO 04/041865, WO 04/041863, WO 04/062551, WO 05/044858, WO 06/40153, WO 06/079372, WO 06/122786, WO 06/122787 and WO 06/122825, relevant portions incorporated herein by reference. As described in these references, Nanobodies (in particular VHH sequences and partially humanized nanobodies), include humanization and/or camelization of the nanobodies, as well as other modifications, parts or fragments, derivatives or “Nanobody fusions”, multivalent constructs (including some non-limiting examples of linker sequences) and different modifications to increase the half-life of the Nanobodies and their preparations can be found e.g. in WO 08/101985 and WO 08/142164, relevant portions incorporated herein by reference.

As used herein, a “subject” may be a mammalian subject. Mammalian subjects include, humans, non-human primates, rodents, (e.g., rats, mice), lagomorphs (e.g., rabbits), ungulates (e.g., cows, sheep, pigs, horses, goats, and the like), etc. In some embodiments, the subject is a human. In some embodiments, the subject is a non-human primate, for example a cynomolgus monkey. In some embodiments, the subject is a companion animal (e.g., cats, dogs).

Native antibodies and immunoglobulins are usually heterotetrameric glycoproteins of about 150,000 daltons, composed of two identical light (L) chains and two identical heavy (H) chains. Each light chain is linked to a heavy chain by at least one covalent disulfide bond, however, the number of disulfide linkages varies between the heavy chains of different immunoglobulin isotypes. Each heavy and light chain also has regularly spaced intrachain disulfide bridges. Each heavy chain has at one end a variable domain (VH) followed by the constant domains. Each light chain has a variable domain at one end (VL) and a constant domain at its other end. The constant domain of the light chain is aligned with the first constant domain of the heavy chain, and the light chain variable domain is aligned with the variable domain of the heavy chain. Particular amino acid residues are believed to form an interface between the light and heavy chain variable domains (Clothia et al., J. Mol. Biol. 186, 651-66, 1985); Novotny and Haber, Proc. Natl. Acad. Sci. USA 82 4592-4596 (1985), relevant portions incorporated herein by reference.

As used herein, an “isolated” antibody is one that has been identified and separated and/or recovered from a component of the environment in which it was produced. Contaminant components of its production environment are materials, which would interfere with diagnostic or therapeutic uses for the antibody, and may include enzymes, hormones, and other proteinaceous or nonproteinaceous solutes. In certain embodiments, the antibody will be purified as measurable by at least three different methods: 1) to greater than 50% by weight of antibody as determined by the Lowry method, such as more than 75% by weight, or more than 85% by weight, or more than 95% by weight, or more than 99% by weight; 2) to a degree sufficient to obtain at least 10 residues of N-terminal or internal amino acid sequence by use of a spinning cup sequentator, such as at least 15 residues of sequence; or 3) to homogeneity by SDS-PAGE under reducing or non-reducing conditions using Coomasie blue or, preferably, silver stain. Isolated antibody includes the antibody in situ within recombinant cells since at least one component of the antibody's natural environment will not be present. Ordinarily, however, isolated antibody will be prepared by at least one purification step.

As used herein, the terms “antibody mutant” or “antibody variant” refer to an amino acid sequence variant of an antibody wherein one or more of the amino acid residues have been modified. Such mutants necessarily have less than 100% sequence identity or similarity with the amino acid sequence having at least 75% amino acid sequence identity or similarity with the amino acid sequence of either the heavy or light chain variable domain of the antibody, such as at least 80%, or at least 85%, or at least 90%, or at least 95, 96, 97, 98, or 99%.

As used herein, the term “variable” in the context of the variable domain of antibodies, refers to the fact that certain portions of the variable domains differ extensively in sequence among antibodies and are used in the binding and specificity of each particular antibody for its particular antigen. However, the variability is not evenly distributed through the variable domains of antibodies. It is concentrated in three segments called complementarity determining regions (CDRs) also known as hypervariable regions both in the light chain and the heavy chain variable domains. There are at least two techniques for determining CDRs: (1) an approach based on cross-species sequence variability (i.e., Kabat et al., Sequences of Proteins of Immunological Interest (National Institute of Health, Bethesda, Md. 1987); and (2) an approach based on crystallographic studies of antigen-antibody complexes (Chothia, C. et al. (1989), Nature 342: 877), or both, that is Chothia plus Kabat. The more highly conserved portions of variable domains are called the framework (FR). The variable domains of native heavy and light chains each comprise four FR regions, largely adopting a s-sheet configuration, connected by three CDRs, which form loops connecting, and in some cases forming part of, the β-sheet structure. The CDRs in each chain are held together in close proximity by the FR regions and, with the CDRs from the other chain, contribute to the formation of the antigen-binding site of antibodies (see Kabat et al.) The constant domains are not involved directly in binding an antibody to its cognate antigen but exhibit various effector function, such as participation of the antibody in antibody-dependent cellular toxicity.

The light chains of antibodies (immunoglobulin) from any vertebrate species can be assigned to one of two clearly distinct types, called kappa and lambda, based on the amino sequences of their constant domain. Depending on the amino acid sequences of the constant domain of their heavy chains, “immunoglobulins” can be assigned to different classes. There are at least five (5) major classes of immunoglobulins: IgA, IgD, IgE, IgG, and IgM, and several of these may be further divided into subclasses (isotypes), e.g., IgG-1, IgG-2, IgG-3, and IgG4; IgA-1 and IgA-2. The subunit structures and three-dimensional configurations of different classes of immunoglobulins are well known.

As used herein, the term “monoclonal antibody” refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to conventional (polyclonal) antibody preparations, which typically include different antibodies directed against different determinants (epitopes), each monoclonal antibody is directed against a single determinant on the antigen. In additional to their specificity, the monoclonal antibodies are advantageous in that they are synthesized by the hybridoma culture, uncontaminated by other immunoglobulins. The modifier “monoclonal” indicates the character of the antibody as being obtained from a substantially homogeneous population of antibodies and is not to be construed as requiring production of the antibody by any particular method. For example, the monoclonal antibodies to be used in accordance with the presently disclosed and claimed invention may be made by the hybridoma method first described by Kohler and Milstein, Nature 256,495 (1975), relevant portions incorporated herein by reference.

All monoclonal antibodies used in accordance with the presently disclosed and claimed invention will be either (1) the result of a deliberate immunization protocol, as described in more detail hereinbelow; or (2) the result of an immune response that results in the production of antibodies naturally in the course of a disease or cancer.

The uses of the monoclonal antibodies of the presently disclosed and claimed invention may require administration of such or similar monoclonal antibody to a subject, such as a human. However, when the monoclonal antibodies are produced in a non-human animal, such as a rodent or chicken, administration of such antibodies to a human patient will normally elicit an immune response, wherein the immune response is directed towards the antibodies themselves. Such reactions limit the duration and effectiveness of such a therapy. To overcome such problem, the monoclonal antibodies of the presently disclosed and claimed invention can be “humanized”, that is, the antibodies are engineered such that antigenic portions thereof are removed and like portions of a human antibody are substituted therefore, while the antibodies' affinity for the amylin receptor is retained. This engineering may only involve a few amino acids, or may include entire framework regions of the antibody, leaving only the complementarity determining regions of the antibody intact. Several methods of humanizing antibodies are known in the art and are disclosed in U.S. Pat. No. 6,180,370, issued to Queen et al on Jan. 30, 2001; U.S. Pat. No. 6,054,927, issued to Brickell on Apr. 25, 2000; U.S. Pat. No. 5,869,619, issued to Studnicka on Feb. 9, 1999; U.S. Pat. No. 5,861,155, issued to Lin on Jan. 19, 1999; U.S. Pat. No. 5,712,120, issued to Rodriquez et al on Jan. 27, 1998; and U.S. Pat. No. 4,816,567, issued to Cabilly et al on Mar. 28, 1989, relevant portions incorporated herein by reference.

Humanized forms of antibodies are chimeric immunoglobulins, immunoglobulin chains or fragments thereof (such as Fab, Fab′, F(ab′)₂, Fv, scFv or other antigen-binding subsequences of antibodies) that are principally comprised of the sequence of a human immunoglobulin, and contain minimal sequence derived from a non-human immunoglobulin. Humanization can be performed following the method of Winter and co-workers (Jones et al., 1986; Riechmann et al., 1988; Verhoeyen et al., 1988), by substituting nonhuman (i.e., rodent, chicken) CDRs or CDR sequences for the corresponding sequences of a human antibody, see, e.g., U.S. Pat. No. 5,225,539. In some instances, Fv framework residues of the human immunoglobulin are replaced by corresponding non-human residues from the donor antibody. Humanized antibodies can also comprise residues that are found neither in the recipient antibody nor in the imported CDR or framework sequences. In general, the humanized antibody will comprise substantially all of, at least one, and typically two, variable domains, in which all or substantially all of the CDR regions correspond to those of a non-human immunoglobulin and all or substantially all of the framework regions are those of a human immunoglobulin consensus sequence. The humanized antibody optimally also will comprise at least a portion of an immunoglobulin constant region (Fc), typically that of a human immunoglobulin.

The presently disclosed and claimed invention further includes the use of fully human monoclonal antibodies cross-reactive against the amylin receptor. Fully human antibodies essentially relate to antibody molecules in which the entire sequence of both the light chain and the heavy chain, including the CDRs, arise from human genes. Such antibodies are termed “human antibodies” or “fully human antibodies” herein. Human monoclonal antibodies can be prepared by, e.g., the trioma technique; the human B-cell hybridoma technique (see Kozbor, et al., Hybridoma, 2:7 (1983)) and the EBV hybridoma technique to produce human monoclonal antibodies (see Cole, et al., PNAS 82:859 (1985)), or as taught herein. Human monoclonal antibodies may be utilized in the practice of the presently disclosed and claimed invention and may be produced by using human hybridomas (see Cote, et al., PNAS 80:2026 (1983)) or by transforming human B-cells with Epstein Barr Virus in vitro (see Cole, et al., 1985), relevant portions incorporated herein by reference.

In addition, human antibodies can be made by introducing human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example but not by way of limitation, in U.S. Pat. Nos. 5,545,807; 5,545,806; 5,569,825; 5,625,126; 5,633,425; 5,661,016, and in Marks et al., J Biol. Chem. 267:16007, (1992); Lonberg et al., Nature, 368:856 (1994); Morrison, 1994; Fishwild et al., Nature Biotechnol. 14:845 (1996); Neuberger, Nat. Biotechnol. 14:826 (1996); and Lonberg and Huszar, Int Rev Immunol. 13:65 (1995), relevant portions incorporated herein by reference.

A method for producing an antibody of interest, such as a human antibody, is disclosed in U.S. Pat. No. 5,916,771, issued to Hori et al. on Jun. 29, 1999, and incorporated herein by reference. It includes introducing an expression vector that contains a nucleotide sequence encoding a heavy chain into one mammalian host cell in culture, introducing an expression vector containing a nucleotide sequence encoding a light chain into another mammalian host cell, and fusing the two cells to form a hybrid cell. The hybrid cell expresses an antibody containing the heavy chain and the light chain.

As used herein, the term “treatment” refers to both therapeutic treatment and prophylactic or preventative measures. Those in need of treatment include those already with the disorder as well as those in which the disorder is to be prevented.

As used herein, the term “disorder” refers to any condition that would benefit from treatment with the polypeptide. This includes chronic and acute disorders or diseases including those infectious or pathological conditions that predispose the mammal to the disorder in question.

An antibody or antibody fragment can be generated with an engineered sequence or glycosylation state to confer preferred levels of activity in antibody-dependent cellular cytotoxicity (ADCC), antibody-dependent cellular phagocytosis (ADCP), antibody-dependent neutrophil phagocytosis (ADNP), or antibody-dependent complement deposition (ADCD) functions as measured by bead-based or cell-based assays or in vivo studies in animal models.

Alternatively, or additionally, it may be useful to combine amino acid modifications with one or more further amino acid modifications that alter complement component Clq binding and/or the complement-dependent cytotoxicity (CDC) function of the Fc region of an IL-23p19 binding molecule. The binding polypeptide of particular interest may be one that binds to Clq and displays complement-dependent cytotoxicity. Polypeptides with pre-existing Clq binding activity, optionally further having the ability to mediate CDC may be modified such that one or both of these activities are enhanced. Amino acid modifications that alter Clq and/or modify its complement-dependent cytotoxicity function are described, for example, in WO/0042072, which is hereby incorporated by reference.

An Fc region of an antibody can be designed to alter the effector function, e.g., by modifying Clq binding and/or FcγR binding and thereby changing complement-dependent cytotoxicity (CDC) activity and/or antibody-dependent cell-mediated cytotoxicity (ADCC) activity. These “effector functions” are responsible for activating or diminishing a biological activity (e.g., in a subject). Examples of effector functions include, but are not limited to: Clq binding; CDC; Fc receptor binding; ADCC; phagocytosis; down-regulation of cell surface receptors (e.g., B cell receptor; BCR), etc. Such effector functions may require the Fc region to be combined with a binding domain (e.g., an antibody variable domain) and can be assessed using various assays (e.g., Fc binding assays, ADCC assays, CDC assays, etc.).

For example, one can generate a variant Fc region of an antibody with improved Clq binding and improved FcγRIII binding (e.g., having both improved ADCC activity and improved CDC activity). Alternatively, if it is desired that effector function be reduced or ablated, a variant Fc region can be engineered with reduced CDC activity and/or reduced ADCC activity. In other embodiments, only one of these activities may be increased, and, optionally, also the other activity reduced (e.g., to generate an Fc region variant with improved ADCC activity, but reduced CDC activity and vice versa).

A single chain variable fragment (scFv) is a fusion of the variable regions of the heavy and light chains of immunoglobulins, linked together with a short (usually serine, glycine) linker. This chimeric molecule retains the specificity of the original immunoglobulin, despite removal of the constant regions and the introduction of a linker peptide. This modification usually leaves the specificity unaltered. These molecules were created historically to facilitate phage display where it is highly convenient to express the antigen-binding domain as a single peptide. Alternatively, scFv can be created directly from subcloned heavy and light chains derived from a hybridoma or B cell. Single chain variable fragments lack the constant Fc region found in complete antibody molecules, and thus, the common binding sites (e.g., protein A/G) used to purify antibodies. These fragments can often be purified/immobilized using Protein L since Protein L interacts with the variable region of kappa light chains.

Flexible linkers generally are comprised of helix- and turn-promoting amino acid residues such as alanine, serine, and glycine. However, other residues can function as well. Phage display can be used to rapidly select tailored linkers for single-chain antibodies (scFvs) from protein linker libraries. A random linker library was constructed in which the genes for the heavy and light chain variable domains were linked by a segment encoding an 18-amino acid polypeptide of variable composition. The scFv repertoire (approx. 1-5×10⁶ different members) is displayed on filamentous phage and subjected to affinity selection with hapten. The population of selected variants exhibited significant increases in binding activity but retained considerable sequence diversity. Sequence analysis revealed a conserved proline in the linker two residues after the VH C terminus and an abundance of arginines and prolines at other positions as the only common features of the selected tethers. In certain embodiments, the antibody fragments are further modified to increase their serum half-life by using modified Fc regions or mutations to the various constant regions, as are known in the art.

In certain embodiments, the antibodies of the present invention are formulated for administration to humans. For example, the antibodies of the present invention can be included in a pharmaceutical composition formulated for an administration that is: intranasal, intrapulmonary, intrabronchial, intravenous, oral, intraadiposal, intraarterial, intraarticular, intracranial, intradermal, intralesional, intramuscular, intrapericardial, intraperitoneal, intrapleural, intravesicular, local, mucosal, parenteral, enteral, subcutaneous, sublingual, topical, transbuccal, transdermal, via inhalation, via injection, in creams, in lipid compositions, via a catheter, via a lavage, via continuous infusion, via infusion, via local delivery, or via localized perfusion, and wherein the composition is a serum, drop, gel, ointment, spray, reservoir, or mist.

As used herein, the term “antigen” refers to a molecule containing one or more epitopes (either linear, conformational or both) that will stimulate a host's immune-system to make a humoral and/or cellular antigen-specific response. The term is used interchangeably with the term “immunogen.” Normally, a B-cell epitope will include at least about 5 amino acids but can be as small as 3-4 amino acids. A T-cell epitope, such as a CTL epitope, will include at least about 7-9 amino acids, and a helper T-cell epitope at least about 12-20 amino acids. Normally, an epitope will include between about 7 and 15 amino acids, such as, 9, 10, 12 or 15 amino acids. The term includes polypeptides, which include modifications, such as deletions, additions and substitutions (generally conservative in nature) as compared to a native sequence, so long as the protein maintains the ability to elicit an immunological response, as defined herein. These modifications may be deliberate, as through site-directed mutagenesis, or may be accidental, such as through mutations of hosts, which produce the antigens.

As used herein, the term “epitope” refers to a specific amino acid sequence or molecule (such as a carbohydrate, small molecule, lipid, etc.) that when present in the proper conformation, provides a reactive site for an antibody (e.g., B cell epitope) or in the case of a peptide to a T cell receptor (e.g., T cell epitope).

Portions of a given polypeptide that include a B-cell epitope can be identified using any number of epitope mapping techniques that are known in the art. (See, e.g., Epitope Mapping Protocols in Methods in Molecular Biology, Vol. 66, Glenn E. Morris, Ed., 1996, Humana Press, Totowa, N.J.). For example, linear epitopes can be determined by, e.g., concurrently synthesizing large numbers of peptides on solid supports, the peptides corresponding to portions of the protein molecule, and reacting the peptides with antibodies while the peptides are still attached to the supports. Such techniques are known in the art and described in, e.g., U.S. Pat. No. 4,708,871; Geysen et al. (1984) Proc. Natl. Acad Sci. USA 81:3998-4002; Geysen et al. (1986) Molec. Immunol. 23:709-715.

As used herein, the term “substantially purified” refers to isolation of a substance (compound, polynucleotide, protein, polypeptide, polypeptide composition) such that the substance comprises the majority percent of the sample in which it resides. Typically, in a sample a substantially purified component comprises 50%, preferably 80%-85%, more preferably 90-95% of the sample. Techniques for purifying polynucleotides and polypeptides of interest are well-known in the art and include, for example, ion-exchange chromatography, affinity chromatography and sedimentation according to density.

As used herein, the term “treatment” refers to both therapeutic treatment and prophylactic or preventative measures. Those in need of treatment include those already with the disorder as well as those in which the disorder is to be prevented.

As used herein, the term “disorder” refers to any condition that would benefit from treatment with the polypeptide. This includes chronic and acute disorders or diseases including those infectious or pathological conditions that predispose the mammal to the disorder in question.

The practice of the present invention employs, unless otherwise indicated, conventional methods of chemistry, biochemistry, molecular biology, immunology and pharmacology, within the skill of the art. Such techniques are explained fully in the literature. See, e.g., Remington's Pharmaceutical Sciences, 18th Edition (Easton, Pa.: Mack Publishing Company, 1990); Methods In Enzymology (S. Colowick and N. Kaplan, eds., Academic Press, Inc.); and Handbook of Experimental Immunology, Vols. I-IV (D. M. Weir and C. C. Blackwell, eds., 1986, Blackwell Scientific Publications); Sambrook, et al., Molecular Cloning: A Laboratory Manual (2nd Edition, 1989); Short Protocols in Molecular Biology, 4th ed. (Ausubel et al. eds., 1999, John Wiley & Sons); Molecular Biology Techniques: An Intensive Laboratory Course, (Ream et al., eds., 1998, Academic Press); PCR (Introduction to Biotechniques Series), 2nd ed. (Newton & Graham eds., 1997, Springer Verlag); Fundamental Virology, Second Edition (Fields & Knipe eds., 1991, Raven Press, New York), relevant portion incorporated herein by reference.

Conservative amino acid substitutions involve replacement of the aliphatic or hydrophobic amino acids Ala, Val, Leu and Ile; replacement of the hydroxyl residues Ser and Thr; replacement of the acidic residues Asp and Glu; replacement of the amide residues Asn and Gln, replacement of the basic residues Lys, Arg, and His; replacement of the aromatic residues Phe, Tyr, and Trp, and replacement of the small-sized amino acids Ala, Ser, Thr, Met, and Gly.

Provided herein are antibodies that bind to the amylin receptor and elicit an agonist response. These antibodies or nanobodies are referred to herein as anti-amylin receptor antibodies or nanobodies. A number of discovery strategies have been employed to obtain the exemplary antibodies of the disclosure, further discussed below.

### Amylin Receptor In-Vitro Selection Strategies.

1. Human Naïve scFv and VHH libraries designed from validated clinical scaffolds.
2. Phage Selections performed starting with engineered soluble Amylin Receptor antigens, followed by cells. Selections using only Amylin receptor expressing cells run in parallel for comparison.
3. Mammalian selections planned for further optimization. Assay development for direct selection based on agonism rather than cell binding.

In some embodiments, the MEM-nanoparticles along with full length antibodies or nanobodies are used to immunize a subject in order to produce antibodies specific to the MEM epitope. Monoclonal hybridomas are then created to produce epitope specific anti-amylin receptor antibodies, binding fragments, or nanobodies. In other embodiments, mouse serum is collected and used to produce in vitro scFv libraries. Followed by phage panning against full length anti-amylin receptor and the MEM-nanoparticle to isolate epitope specific clones. In other embodiments, antibodies are generated using phage panning of a naïve antibody library against full length anti-amylin receptor antibodies, binding fragments, or nanobodies. In other embodiments, humanized anti-amylin receptor CDRs are generated from AI-model predictions based on reference antibodies. Mammalian display libraries are then created, and single cell sorted to select for epitope specific anti-amylin receptor antibodies, binding fragments, or nanobodies.

The skilled artisan will recognize that antibodies which exhibit little or no binding to a target antigen can be described as having a low affinity, and a high equilibrium dissociation constant (KD) for the target antigen. The skilled artisan will also recognize that antibodies which exhibit little or no binding to a collective assembly of target antigenic epitopes can be described as having a low avidity, and a high equilibrium dissociation constant (KD) for the collective assembly of target antigenic epitopes.

In some embodiments, provided herein are anti-amylin receptor antibodies, binding fragments, or nanobodies having a binding affinity (KD) to amylin receptor of about 5 μM to about 5 pM, about 1 μM to about 5 pM, about 0.5 μM to about 5 pM, about 0.1 μM to about 5 pM, about 50 nM to about 5 pM, about 10 nM to about 5 pM, about 5 nM to about 5 pM, about 1 nM to about 5 pM, about 0.5 nM to about 5 pM, about 0.1 nM to about 5 pM, about 50 pM to about 5 pM, about 10 pM to about 5 pM.

In some embodiments, the anti-amylin receptor antibodies, binding fragments, or nanobodies have a binding avidity (KD) to amylin receptor of about 500 nM to about 0.1 pM, about 100 nM to about 0.1 pM, about 50 nM to about 0.1 pM, about 10 nM to about 0.1 pM, about 5 nM to about 0.1 pM, about 1 nM to about 0.1 pM, about 0.5 nM to about 0.1 pM, about 0.1 nM to about 0.1 pM, about 50 pM to about 0.1 pM, about 10 pM to about 0.1 pM, about 5 pM to about 0.1 pM, about 1 pM to about 0.1 pM, about 0.5 pM to about 0.1 pM.

In some embodiments, the anti-amylin receptor antibodies, binding fragments, or nanobodies have a half maximal effective concentration (EC50) to amylin receptor of about 500 nM to about 0.001 nM, about 100 nM to about 0.001 nM, about 50 nM to about 0.001 nM, about 10 nM to about 0.001 nM, about 5 nM to about 0.001 nM, about 1 nM to about 0.001 nM, about 0.5 nM to about 0.001 nM, about 0.1 nM to about 0.001 nM, about 0.05 nM to about 0.001 nM, about 0.01 nM to about 0.001 nM, about 0.005 nM to about 0.001 nM.

The skilled artisan will recognize that binding specificity may be determined through a series of competition binding paradigms, in which a desired antibody demonstrates its ability to prevent binding of a known refence antibody to its target epitope at varying concentrations.

In some embodiments, the anti-amylin receptor antibodies, binding fragments, or nanobodies agonist antibody is a full-length antibody (referring to an antibody with two heavy and two light chains attached to the Fc domain, giving a ‘Y’ shape). In some embodiments the Fc domain (or simply referred to as an Fc) is a human Fc domain. In some embodiments, the Fc domain of anti-amylin receptor antibodies, or binding fragments is from a human IgG1, human IgG2, human IgG3, or human IgG4.

### Engineered Epitope Design for Amylin Receptor.

Engineered epitopes were developed to reflect solvent-accessible, conformationally stable epitopes derived from the extracellular domains of the human Amylin receptor heterodimers (AMY1 or AMY3), comprising combinations of Calcitonin Receptor (CTR) and Receptor Activity-Modifying Proteins (RAMPs). Epitope regions were selected based on predicted structural accessibility and immunogenic potential.

Peptide scaffolds were computationally designed using the iBio Engineered Epitope machine learning engine to support the native sequence and structural features of these epitopes. Design blueprints specified fixed epitope residues and tunable scaffold regions. Optimization prioritized: (1) structural fidelity to the native Amylin receptor epitope, (2) overall scaffold stability, and (3) solubility. Scaffold size was iteratively reduced while maintaining all design constraints. Final epitope designs were synthesized as biotinylated peptides for in-vitro selection and screening workflows.

### In-Vitro Antibody Selections.

3-4 rounds of in-vitro phage display selection were performed using a naïve fully human scFv library with an input of (5×10¹² virions per round). Selections employed streptavidin-coated magnetic beads for target capture and negative depletion.

In the first round, biotinylated PSR (irrelevant scaffold) can be used for negative selection. The phage library was incubated with streptavidin beads, and unbound phage was retained. Positive selection was performed using either: (1) biotinylated engineered Amylin receptor epitopes or (2) Expi293F cells transiently transfected with human AMY1 or AMY3 heterodimer constructs (CTR+RAMP1 or RAMP3). The same transfected cells used in the cell binding assay were used here, with expression validated by fluorescent reporter.

For cell-based selections, transfected cells were pelleted between wash and elution steps at 300×g. Untransfected Expi293F cells were used for negative selection to deplete phages that bound non-specifically to host cell components.

After incubation with the positive selection agent (engineered epitope or transfected cells), phages were captured (either by streptavidin beads for epitopes or direct recovery from cell pellets), washed, and eluted for amplification in E. coli.

Subsequent rounds repeated the negative selection with PSR or untransfected cells, followed by positive selection with biotinylated Amylin receptor epitopes or transfected cells. Bound phages were recovered and amplified for downstream characterization.

Phagemids were recovered from the amplified pool for sequence analysis. Select scFvs were subcloned into mammalian expression vectors for functional characterization.

### Antibody Expression and Purification.

Antibody expression constructs were transfected into ExpiCHO-S cells using the ExpiFectamine CHO Transfection Kit (ThermoFisher, Cat #A29129). Cultures were maintained for 6-12 days, then harvested by centrifugation and 0.2 μm filtration. Antibodies were purified from the supernatant using Protein A affinity resin (HiTrap MabSelect SuRe; Cytiva, Cat #GE11-0034-93) and desalted into PBS or other appropriate buffer.

### Cell Binding Assay Using AMY1/AMY3-Transfected Cells.

Expi293F cells were transiently transfected with plasmids encoding the human AMY1 or AMY3 heterodimer (CTR+RAMP1 or RAMP3), each fused to a fluorescent reporter to enable quantification of receptor expression.

Transfected cells were incubated in assay buffer (PBS+2% FBS+2 mM EDTA) and seeded in 96-well plates. Following incubation with titrated concentrations of antibody bound antibody was detected with species-specific Alexa Fluor 647-conjugated secondary antibodies.

### Label-Free Kinetics of Amylin Receptor Binding by SPR.

Binding kinetics to the Amylin receptor engineered epitopes were measured using a Carterra LSA SPR system. Antibodies were captured on an anti-human Fc-functionalized HC200M chip in HBST+0.05% BSA buffer.

Epitopes were injected in increasing concentrations. Each cycle consisted of baseline, association, and dissociation phases. Chip surfaces were regenerated using 0.1M glycine (pH 2.0). Association/dissociation constants were calculated using LSA Kinetics software.

### Anti-Amylin Receptor Antibodies, Binding Fragments, or Nanobodies Sequences.

Provided herein are sequences for exemplary anti-amylin receptor antibodies, binding fragments, or nanobodies sequences agonist antibodies of the disclosure. Included are complementarity determining region (CDR) sequences and the variable heavy and light domain sequences (VH, VL) that constitute the amylin receptor antigen binding domains of the disclosure. The discovery of these antibodies is detailed in the Examples section.

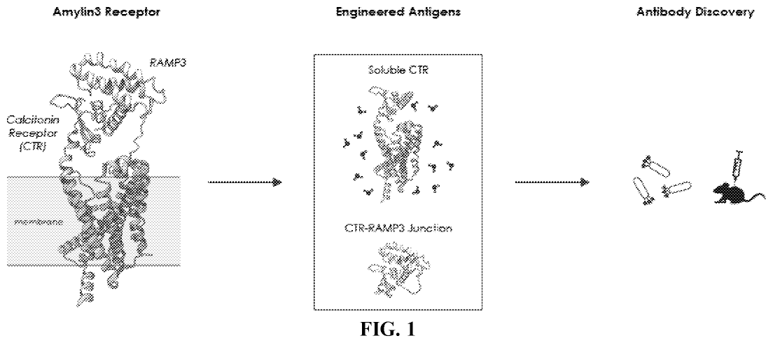

FIG. 1 illustrates the design of the engineered antigens designed for Amylin3 Receptor.

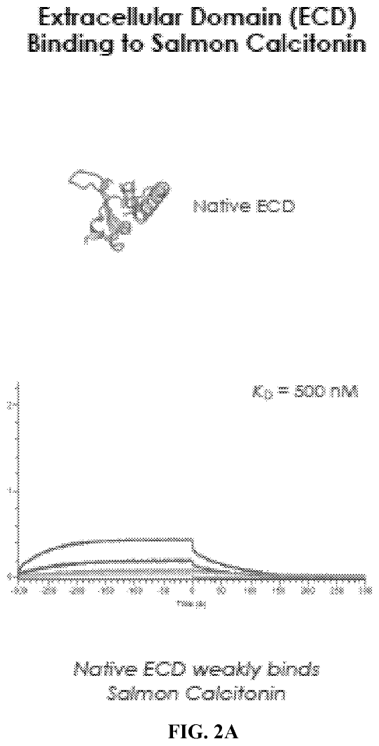

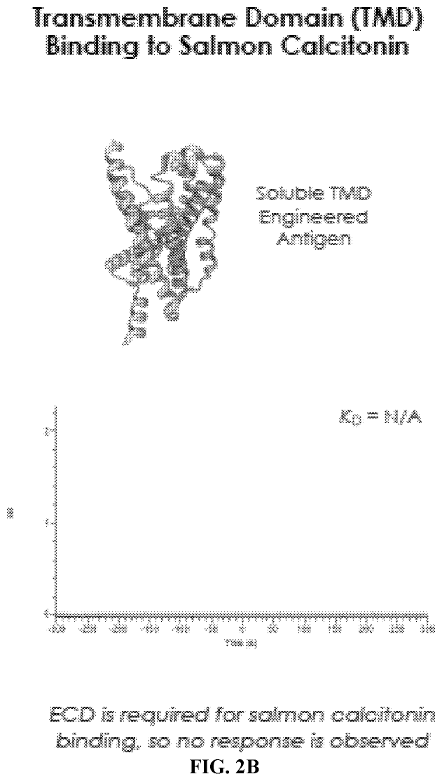

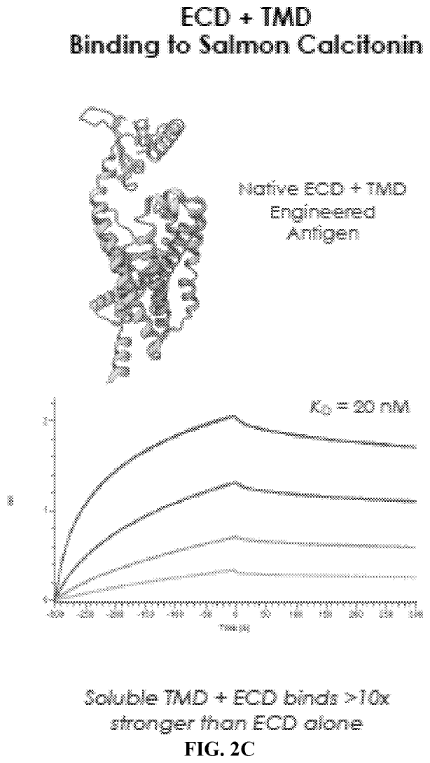

FIGS. 2A to 2C show the validation using Soluble Calcitonin Receptor Validated by Salmon Calcitonin Binding. FIG. 2A shows the binding to the Extracellular Domain (ECD) Binding to Salmon Calcitonin, FIG. 2B shows the binding to the Transmembrane Domain (TMD) Binding to Salmon Calcitonin, and FIG. 2C shows the binding to the ECD+TMD Binding to Salmon Calcitonin.

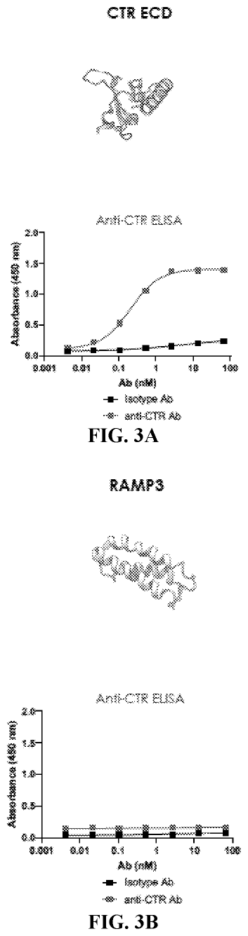

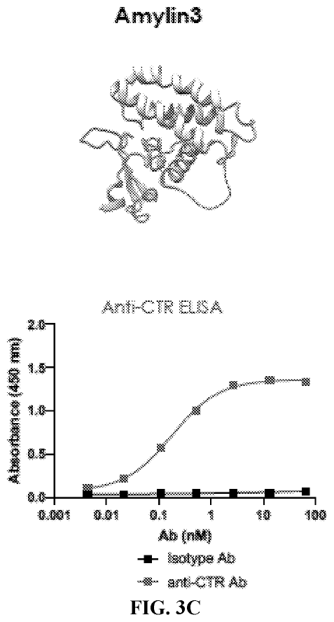

FIGS. 3A to 3C show the validation which shows that the CTR-RAMP3 (Amylin3) Engineered Antigen Binds Anti-CTR. FIG. 3A binding to the CTR extracellular domain (ECD), FIG. 3B binding to RAMP3, and FIG. 3C binding to Amylin 3.

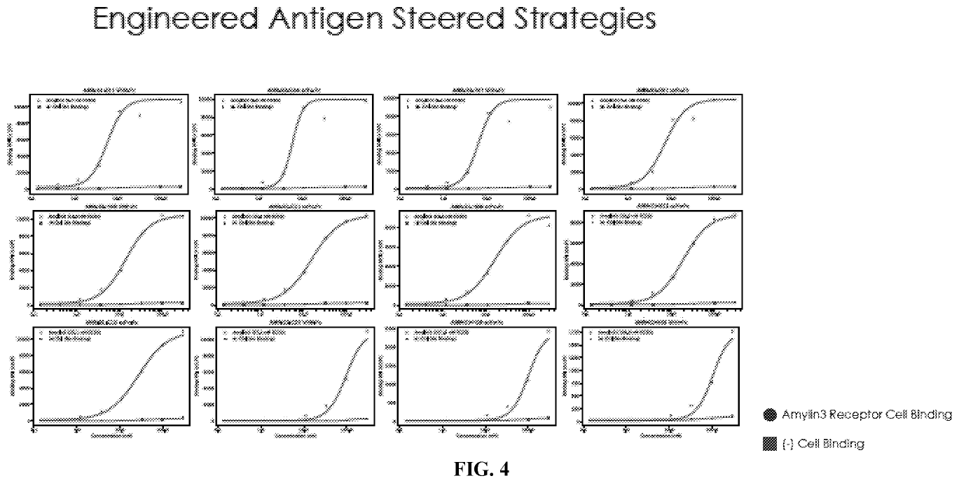

FIG. 4 shows the results from Engineered Antigen Steered Strategies Yield Receptor Specific Cell Binders using engineered antigen steered strategies.

As referred below, a light chain variable (VL) domain CDR1 region is referred to as CDR-L1; a VL CDR2 region is referred to as CDR-L2; a VL CDR3 region is referred to as CDR-L3; a heavy chain variable (VH) domain CDR1 region is referred to as CDR-H1; a VH CDR2 region is referred to as CDR-H2; and a VH CDR3 region is referred to as CDR-H3. Table 1 provides exemplary CDR combinations of antibodies of the disclosure. The skilled artisan will understand that framework regions can be modified, as such, the present disclosure includes sequences that have at least 91, 92, 93, 94, 95, 96, 97, 98, 99, or 100 percent sequence identity to the sequences herein.

TABLE 1
Exemplary Amylin Receptor Agonist Antibody CDR Combinations.

| | VH_cdr1_ | VH_cdr2_ | VH_cdr3_ | VL_cdr1_ | VL_cdr2_ | VL_cdr3_ |
|---|---|---|---|---|---|---|
| AbID | aa | aa | aa | aa | aa | aa |
| AMR-02-G04 | GFTFE | IYANAA | AKDQSILGIY | QSVGHY | TSS | QQSHSSPWT |
| | RYA | GT | GNAMDY | | | |
| SEQ ID NO: | 8 | 10 | 12 | 22 | 24 | 26 |
| AMR-02-E01 | GFTFE | IYANAA | AKDQSILGIY | QSVGHY | ATS | QQSYGNPR |
| | RYA | GT | GNAMDY | | | T |
| SEQ ID NO: | 42 | 44 | 46 | 56 | 58 | 60 |
| AMR-01-C11 | GFTFS | ISGSGVN | AKDQSILGIY | QSVGHY | TSS | QQSHSSPWT |
| | SYG | A | GNAMDY | | | |
| SEQ ID NO: | 76 | 78 | 80 | 90 | 92 | 94 |
| AMR-01-B12 | GLSFS | LSASGG | ARDRGRYGA | QSISSY | ATS | QQSYGNPR |
| | SHA | RT | ESAYPVRAF | | | T |
| | | | DP | | | |
| SEQ ID NO: | 110 | 112 | 114 | 124 | 126 | 128 |
| AMR-01-C10 | GFTFE | IYANAA | AKDQSILGIY | QPISVY | AAS | QQTYSTPHT |
| | RYA | GT | GNAMDY | | | |
| SEQ ID NO: | 144 | 146 | 148 | 158 | 160 | 162 |
| AMR-01-H09 | GFSL | ISGGGG | ARTGPGIVG | QSITKY | SAS | QQSYSAPYS |
| | RNYA | RT | RVSGAFDV | | | |
| SEQ ID NO: | 178 | 180 | 182 | 192 | 194 | 196 |
| AMR-02-E11 | GFTFS | IYSGGST | ARRRSWWS | Note: | | |
| | HYA | | NRDRYHYN | VHHs only | | |
| | | | AFDY | have one | | |
| | | | | variable | | |
| | | | | domain | | |
| SEQ ID NO: | 210 | 212 | 214 | | | |
| AMR-02-C07 | GFTFS | IYSGGST | ARRRSWWS | | | |
| | GYA | | NRDRYHYN | | | |
| | | | AFDY | | | |
| SEQ ID NO: | 228 | 230 | 232 | | | |
| AMR-02-F07 | GFSFS | ISRSGGG | ARDAGGWG | | | |
| | SYL | T | MDV | | | |
| SEQ ID NO: | 246 | 248 | 250 | | | |
| AMR-02-H09 | GFSFS | ISGSGAV | ARDAGGWG | | | |
| | SYL | T | MDV | | | |
| SEQ ID NO: | 264 | 266 | 268 | | | |
| AMR-02-D07 | GFTFS | ITGSGAT | AKGHGDWY | | | |
| | NYA | T | GSGIDI | | | |
| SEQ ID NO: | 282 | 284 | 286 | | | |
| AMR-02-H08 | GFTF | ISGSGGT | ARERLGYPW | | | |
| | NDYA | T | TFDV | | | |
| SEQ ID NO: | 300 | 302 | 304 | | | |

### Exemplary Anti-Amylin Receptor Agonist Antibodies—Variable Region Sequences

The term variable domain and variable region are used interchangeably and refer to the portions of the light and heavy chains of an antibody that include the complementarity determining regions and framework regions (FRs).

Table 2 provides amino acid sequences for the variable domains of exemplary anti-amylin receptor agonist antibodies and nanobodies of the disclosure, including amino acid and nucleic acid sequences.

| | VH | | VL | |
|---|---|---|---|---|
| | Sequence | VH | Sequence | VL |
| AbID | AA | Sequence NT | AA | Sequence NT |
| AMR-02- | EVQLLESG | GAGGTGCAGCTC | DIQMTQSP | GATATTCAGATG |
| G04 | GGLVQPG | TTAGAAAGCGGA | SSLSASVG | ACCCAGAGCCCA |
| | GSLRLSCA | GGTGGCTTGGTT | DRVTITCR | TCCTCCCTGTCA |
| | ASGFTFER | CAGCCTGGCGGT | ASQSVGH | GCTTCTGTAGGA |
| | YAMTWVR | AGTCTAAGACTG | YLNWYQQ | GACCGCGTTACC |
| | QAPGKGLE | TCCTGTGCAGCT | KPGKAPKL | ATCACCTGTCGT |
| | WVSGIYAN | TCTGGCTTCACTT | LIYTSSNL | GCCTCCCAGAGC |
| | AAGTDYA | TTGAACGCTATG | QRGVPSRF | GTGGGCCACTAC |
| | DSVKGRFT | CCATGACCTGGG | SGSGSGTD | CTGAACTGGTAT |
| | ISRDNSKN | TCCGTCAAGCAC | FTLTISSLQ | CAGCAGAAGCCT |
| | TLYLQMNS | CAGGTAAGGGTC | PEDFATYY | GGTAAGGCACCG |
| | LRAEDTAV | TGGAATGGGTGT | CQQSHSSP | AAGCTGCTGATC |
| | YYCAKDQ | CTGGTATCTACG | WTFGGGT | TATACCTCCTCT |
| | SILGIYGNA | CCAACGCAGCGG | KVEIK | AACCTGCAACGT |
| | MDYWGQG | GCACCGATTATG | | GGCGTCCCGTCA |
| | TLVTVSS | CCGATAGCGTGA | | CGCTTTAGTGGC |
| | | AAGGACGCTTCA | | TCTGGTTCTGGG |
| | | CCATCAGCAGGG | | ACCGACTTCACT |
| | | ACAATTCCAAGA | | CTCACCATTAGC |
| | | ACACGCTGTACC | | AGTCTGCAACCG |
| | | TGCAAATGAACA | | GAGGACTTCGCG |
| | | GCCTGCGAGCCG | | ACATACTATTGC |
| | | AGGATACGGCCG | | CAGCAGAGCCAC |
| | | TGTATTACTGTG | | TCTTCCCCGTGG |
| | | CTAAAGATCAAA | | ACTTTTGGAGGC |
| | | GCATCCTGGGAA | | GGTACCAAGGTG |
| | | TCTATGGCAACG | | GAGATCAAA |
| | | CGATGGACTATT | | |
| | | GGGGCCAGGGA | | |
| | | ACCCTGGTCACC | | |
| | | GTCTCCTCA | | |
| SEQ ID | 3 | 4 | 5 | 6 |
| NO: | | | | |
| AMR-02- | EVQLLESG | GAGGTGCAGCTC | DIQMTQSP | GATATTCAGATG |
| E01 | GGLVQPG | TTAGAAAGCGGA | SSLSASVG | ACCCAGAGCCCA |
| | GSLRLSCA | GGTGGCTTGGTT | DRVTITCR | TCCTCCCTGTCA |
| | ASGFTFER | CAGCCTGGCGGT | ASQSVGH | GCTTCTGTAGGA |
| | YAMTWVR | AGTCTAAGACTG | YLNWYQQ | GACCGCGTTACC |
| | QAPGKGLE | TCCTGTGCAGCT | KPGKAPKL | ATCACCTGTCGT |
| | WVSGIYAN | TCTGGCTTCACTT | LIYATSTL | GCCTCCCAGAGC |
| | AAGTDYA | TTGAACGCTATG | HSGVPSRF | GTGGGCCACTAC |
| | DSVKGRFT | CCATGACCTGGG | SGSGSGTD | CTGAACTGGTAT |
| | ISRDNSKN | TCCGTCAAGCAC | FTLTISSLQ | CAGCAGAAGCCT |
| | TLYLQMNS | CAGGTAAGGGTC | PEDFATYY | GGTAAGGCACCG |
| | LRAEDTAV | TGGAATGGGTGT | CQQSYGN | AAGCTGCTGATC |
| | YYCAKDQ | CTGGTATCTACG | PRTFGGGT | TATGCCACCAGC |
| | SILGIYGNA | CCAACGCAGCGG | KVEIK | ACTCTGCACTCT |
| | MDYWGQG | GCACCGATTATG | | GGCGTCCCGTCA |
| | TLVTVSS | CCGATAGCGTGA | | CGCTTTAGTGGC |
| | | AAGGACGCTTCA | | TCTGGTTCTGGG |
| | | CCATCAGCAGGG | | ACCGACTTCACT |
| | | ACAATTCCAAGA | | CTCACCATTAGC |
| | | ACACGCTGTACC | | AGTCTGCAACCG |
| | | TGCAAATGAACA | | GAGGACTTCGCG |
| | | GCCTGCGAGCCG | | ACATACTATTGC |
| | | AGGATACGGCCG | | CAGCAGAGCTAT |
| | | TGTATTACTGTG | | GGTAATCCGCGC |
| | | CTAAAGATCAAA | | ACCTTTGGAGGC |
| | | GCATCCTGGGAA | | GGTACCAAGGTG |
| | | TCTATGGCAACG | | GAGATCAAA |
| | | CGATGGACTATT | | |
| | | GGGGCCAGGGA | | |
| | | ACCCTGGTCACC | | |
| | | GTCTCCTCA | | |
| SEQ ID | 37 | 38 | 39 | 40 |
| NO: | | | | |
| AMR-01- | EVQLLESG | GAGGTGCAGCTC | DIQMTQSP | GATATTCAGATG |
| C11 | GGLVQPG | TTAGAAAGCGGA | SSLSASVG | ACCCAGAGCCCA |
| | GSLRLSCA | GGTGGCTTGGTT | DRVTITCR | TCCTCCCTGTCA |
| | ASGFTFSS | CAGCCTGGCGGT | ASQSVGH | GCTTCTGTAGGA |
| | YGLAWVR | AGTCTAAGACTG | YLNWYQQ | GACCGCGTTACC |
| | QAPGKGLE | TCCTGTGCAGCT | KPGKAPKL | ATCACCTGTCGT |
| | WVSAISGS | TCTGGTTTTACCT | LIYTSSNL | GCCTCCCAGAGC |
| | GVNAHYA | TCAGCTCTTATG | QRGVPSRF | GTGGGCCACTAC |
| | DSVKGRFT | GCCTGGCTTGGG | SGSGSGTD | CTGAACTGGTAT |
| | ISRDNSKN | TCCGTCAAGCAC | FTLTISSLQ | CAGCAGAAGCCT |
| | TLYLQMNS | CAGGTAAGGGTC | PEDFATYY | GGTAAGGCACCG |
| | LRAEDTAV | TGGAATGGGTGT | CQQSHSSP | AAGCTGCTGATC |
| | YYCAKDQ | CTGCAATCTCTG | WTFGGGT | TATACCTCCTCT |
| | SILGIYGNA | GCTCCGGTGTGA | KVEIK | AACCTGCAACGT |
| | MDYWGQG | ACGCTCACTATG | | GGCGTCCCGTCA |
| | TLVTVSS | CCGATAGCGTGA | | CGCTTTAGTGGC |
| | | AAGGACGCTTCA | | TCTGGTTCTGGG |
| | | CCATCAGCAGGG | | ACCGACTTCACT |
| | | ACAATTCCAAGA | | CTCACCATTAGC |
| | | ACACGCTGTACC | | AGTCTGCAACCG |
| | | TGCAAATGAACA | | GAGGACTTCGCG |
| | | GCCTGCGAGCCG | | ACATACTATTGC |
| | | AGGATACGGCCG | | CAGCAGAGCCAC |
| | | TGTATTACTGTG | | TCTTCCCCGTGG |
| | | CTAAAGATCAAA | | ACTTTTGGAGGC |
| | | GCATCCTGGGAA | | GGTACCAAGGTG |
| | | TCTATGGCAACG | | GAGATCAAA |
| | | CGATGGACTATT | | |
| | | GGGGCCAGGGA | | |
| | | ACCCTGGTCACC | | |
| | | GTCTCCTCA | | |
| SEQ ID | 71 | 72 | 73 | 74 |
| NO: | | | | |
| AMR-01- | EVQLLESG | GAGGTGCAGCTC | DIQMTQSP | GATATTCAGATG |
| B12 | GGLVQPG | TTAGAAAGCGGA | SSLSASVG | ACCCAGAGCCCA |
| | GSLRLSCA | GGTGGCTTGGTT | DRVTITCR | TCCTCCCTGTCA |
| | ASGLSFSS | CAGCCTGGCGGT | ASQSISSYL | GCTTCTGTAGGA |
| | HAMSWVR | AGTCTAAGACTG | NWYQQKP | GACCGCGTTACC |
| | QAPGKGLE | TCCTGTGCAGCT | GKAPKLLI | ATCACCTGTCGT |
| | WVSGLSAS | TCTGGCCTGTCTT | YATSTLHS | GCCTCCCAATCT |
| | GGRTYYA | TTAGCAGCCACG | GVPSRFSG | ATTAGCAGCTAC |
| | DSVKGRFT | CTATGTCATGGG | SGSGTDFT | CTGAACTGGTAT |
| | ISRDNSKN | TCCGTCAAGCAC | LTISSLQPE | CAGCAGAAGCCT |
| | TLYLQMNS | CAGGTAAGGGTC | DFATYYC | GGTAAGGCACCG |
| | LRAEDTAV | TGGAATGGGTGT | QQSYGNP | AAGCTGCTGATC |
| | YYCARDR | CTGGTCTGTCTG | RTFGGGTK | TATGCCACCAGC |
| | GRYGAESA | CAAGCGGAGGCC | VEIK | ACTCTGCACTCT |
| | YPVRAFDP | GCACCTACTATG | | GGCGTCCCGTCA |
| | WGQGTLV | CCGATAGCGTGA | | CGCTTTAGTGGC |
| | TVSS | AAGGACGCTTCA | | TCTGGTTCTGGG |
| | | CCATCAGCAGGG | | ACCGACTTCACT |
| | | ACAATTCCAAGA | | CTCACCATTAGC |
| | | ACACGCTGTACC | | AGTCTGCAACCG |
| | | TGCAAATGAACA | | GAGGACTTCGCG |
| | | GCCTGCGAGCCG | | ACATACTATTGC |
| | | AGGATACGGCCG | | CAGCAGAGCTAT |
| | | TGTATTACTGTG | | GGTAATCCGCGC |
| | | CCAGGGATCGGG | | ACCTTTGGAGGC |
| | | GTAGGTACGGGG | | GGTACCAAGGTG |
| | | CCGAATCCGCGT | | GAGATCAAA |
| | | ATCCCGTGCGGG | | |
| | | CCTTTGACCCTT | | |
| | | GGGGCCAGGGA | | |
| | | ACCCTGGTCACC | | |
| | | GTCTCCTCA | | |
| SEQ ID | 105 | 106 | 107 | 108 |
| NO: | | | | |
| AMR-01- | EVQLLESG | GAGGTGCAGCTC | DIQMTQSP | GATATTCAGATG |
| C10 | GGLVQPG | TTAGAAAGCGGA | SSLSASVG | ACCCAGAGCCCA |
| | GSLRLSCA | GGTGGCTTGGTT | DRVTITCR | TCCTCCCTGTCA |
| | ASGFTFER | CAGCCTGGCGGT | ASQPISVY | GCTTCTGTAGGA |
| | YAMTWVR | AGTCTAAGACTG | LHWYQQK | GACCGCGTTACC |
| | QAPGKGLE | TCCTGTGCAGCT | PGKAPKLL | ATCACCTGTCGT |
| | WVSGIYAN | TCTGGCTTCACTT | IYAASSLQ | GCCTCCCAGCCG |
| | AAGTDYA | TTGAACGCTATG | SGVPSRFS | ATTAGCGTGTAC |
| | DSVKGRFT | CCATGACCTGGG | GSGSGTDF | CTGCACTGGTAT |
| | ISRDNSKN | TCCGTCAAGCAC | TLTISSLQP | CAGCAGAAGCCT |
| | TLYLQMNS | CAGGTAAGGGTC | EDFATYYC | GGTAAGGCACCG |
| | LRAEDTAV | TGGAATGGGTGT | QQTYSTPH | AAGCTGCTGATC |
| | YYCAKDQ | CTGGTATCTACG | TFGGGTK | TATGCCGCATCC |
| | SILGIYGNA | CCAACGCAGCGG | VEIK | AGCCTCCAGTCT |
| | MDYWGQG | GCACCGATTATG | | GGCGTCCCGTCA |
| | TLVTVSS | CCGATAGCGTGA | | CGCTTTAGTGGC |
| | | AAGGACGCTTCA | | TCTGGTTCTGGG |
| | | CCATCAGCAGGG | | ACCGACTTCACT |
| | | ACAATTCCAAGA | | CTCACCATTAGC |
| | | ACACGCTGTACC | | AGTCTGCAACCG |
| | | TGCAAATGAACA | | GAGGACTTCGCG |
| | | GCCTGCGAGCCG | | ACATACTATTGC |
| | | AGGATACGGCCG | | CAGCAGACCTAT |
| | | TGTATTACTGTG | | AGCACCCCGCAT |
| | | CTAAAGATCAAA | | ACTTTTGGAGGC |
| | | GCATCCTGGGAA | | GGTACCAAGGTG |
| | | TCTATGGCAACG | | GAGATCAAA |
| | | CGATGGACTATT | | |
| | | GGGGCCAGGGA | | |
| | | ACCCTGGTCACC | | |
| | | GTCTCCTCA | | |
| SEQ ID | 139 | 140 | 141 | 142 |
| NO: | | | | |
| AMR-01- | EVQLLESG | GAGGTGCAGCTC | DIQMTQSP | GATATTCAGATG |
| H09 | GGLVQPG | TTAGAAAGCGGA | SSLSASVG | ACCCAGAGCCCA |
| | GSLRLSCA | GGTGGCTTGGTT | DRVTITCR | TCCTCCCTGTCA |
| | ASGFSLRN | CAGCCTGGCGGT | TSQSITKY | GCTTCTGTAGGA |
| | YAVSWVR | AGTCTAAGACTG | LNWYQQK | GACCGCGTTACC |
| | QAPGKGLE | TCCTGTGCAGCT | PGKAPKLL | ATCACCTGTCGT |
| | WVSTISGG | TCTGGCTTTAGC | IYSASSLQS | ACATCCCAGAGC |
| | GGRTHYA | TTGCGCAACTAT | GVPSRFSG | ATCACTAAATAC |
| | DSVKGRFT | GCTGTGTCCTGG | SGSGTDFT | CTGAACTGGTAT |
| | ISRDNSKN | GTCCGTCAAGCA | LTISSLQPE | CAGCAGAAGCCT |
| | TLYLQMNS | CCAGGTAAGGGT | DFATYYC | GGTAAGGCACCG |
| | LRAEDTAV | CTGGAATGGGTG | QQSYSAPY | AAGCTGCTGATC |
| | YYCARTGP | TCTACCATCTCC | SFGGGTKV | TATTCTGCCTCC |
| | GIVGRVSG | GGTGGCGGTGGC | EIK | AGCCTGCAAAGC |
| | AFDVWGQ | CGCACTCACTAT | | GGCGTCCCGTCA |
| | GTLVTVSS | GCCGATAGCGTG | | CGCTTTAGTGGC |
| | | AAAGGACGCTTC | | TCTGGTTCTGGG |
| | | ACCATCAGCAGG | | ACCGACTTCACT |
| | | GACAATTCCAAG | | CTCACCATTAGC |
| | | AACACGCTGTAC | | AGTCTGCAACCG |
| | | CTGCAAATGAAC | | GAGGACTTCGCG |
| | | AGCCTGCGAGCC | | ACATACTATTGC |
| | | GAGGATACGGCC | | CAGCAGTCTTAC |
| | | GTGTATTACTGT | | TCCGCACCGTAT |
| | | GCACGTACTGGC | | AGCTTTGGAGGC |
| | | CCTGGGATCGTC | | GGTACCAAGGTG |
| | | GGACGGGTTTCA | | GAGATCAAA |
| | | GGCGCTTTTGAT | | |
| | | GTCTGGGGCCAG | | |
| | | GGAACCCTGGTC | | |
| | | ACCGTCTCCTCA | | |
| SEQ ID | 173 | 174 | 175 | 176 |
| NO: | | | | |
| AMR-02- | EVQLVESG | GAGGTTCAGTTG | No VL | No VL |
| E11 | GGLVQPG | GTCGAGTCTGGT | | |
| | GSLRLSCA | GGTGGGCTCGTG | | |
| | ASGFTFSH | CAACCCGGAGGC | | |
| | YAMSWFR | TCCCTGAGGTTG | | |
| | QAPGKGRE | AGCTGCGCCGCT | | |
| | LVAVIYSG | TCAGGCTTTACA | | |
| | GSTYYPDS | TTCAGCCACTAC | | |
| | VEGRFTISR | GCCATGAGCTGG | | |
| | DNAKKML | TTCAGACAAGCA | | |
| | YLQMNSL | CCTGGTAAGGGC | | |
| | RAEGDTA | AGAGAACTGGTG | | |
| | VYYCARR | GCTGTGATCTAC | | |
| | RSWWSNR | TCCGGAGGCAGC | | |
| | DRYHYNA | ACCTATTACCCC | | |
| | FDYWGQG | GATTCCGTGGAG | | |
| | TLVTVSS | GGACGGTTTACT | | |
| | | ATTTCAAGGGAC | | |
| | | AACGCTAAGAAA | | |
| | | ATGCTATACCTT | | |
| | | CAGATGAATAGT | | |
| | | TTGAGGGCAGAG | | |
| | | GGAGATACGGCC | | |
| | | GTGTATTACTGT | | |
| | | GCTCGCAGACGG | | |
| | | AGCTGGTGGTCA | | |
| | | AACCGTGATCGC | | |
| | | TATCACTATAAC | | |
| | | GCCTTTGACTAC | | |
| | | TGGGGCCAGGGA | | |
| | | ACCCTGGTCACC | | |
| | | GTCTCCTCA | | |
| SEQ ID | 207 | 208 | | |
| NO: | | | | |
| AMR-02- | EVQLVESG | GAGGTTCAGTTG | No VL | No VL |
| C07 | GGLVQPG | GTCGAGTCTGGT | | |
| | GSLRLSCA | GGTGGGCTCGTG | | |
| | ASGFTFSG | CAACCCGGAGGC | | |
| | YAMSWFR | TCCCTGAGGTTG | | |
| | QAPGKGRE | AGCTGCGCCGCT | | |
| | LVAVIYSG | TCAGGGTTCACC | | |
| | GSTYYPDS | TTTTCCGGCTAC | | |
| | VEGRFTISR | GCCATGAGCTGG | | |
| | DNAKKML | TTCAGACAAGCA | | |
| | YLQMNSL | CCTGGTAAGGGC | | |
| | RAEGDTA | AGAGAACTGGTG | | |
| | VYYCARR | GCTGTGATCTAC | | |
| | RSWWSNR | TCCGGAGGCAGC | | |
| | DRYHYNA | ACCTATTACCCC | | |
| | FDYWGQG | GATTCCGTGGAG | | |
| | TLVTVSS | GGACGGTTTACT | | |
| | | ATTTCAAGGGAC | | |
| | | AACGCTAAGAAA | | |
| | | ATGCTATACCTT | | |
| | | CAGATGAATAGT | | |
| | | TTGAGGGCAGAG | | |
| | | GGAGATACGGCC | | |
| | | GTGTATTACTGT | | |
| | | GCTCGCAGACGG | | |
| | | AGCTGGTGGTCA | | |
| | | AACCGTGATCGC | | |
| | | TATCACTATAAC | | |
| | | GCCTTTGACTAC | | |
| | | TGGGGCCAGGGA | | |
| | | ACCCTGGTCACC | | |
| | | GTCTCCTCA | | |
| SEQ ID | 225 | 226 | | |
| NO: | | | | |
| AMR-02- | EVQLVESG | GAGGTTCAGTTG | No VL | No VL |
| F07 | GGLVQPG | GTCGAGTCTGGT | | |
| | GSLRLSCA | GGTGGGCTCGTG | | |
| | ASGFSFSS | CAACCCGGAGGC | | |
| | YLMAWFR | TCCCTGAGGTTG | | |
| | QAPGKGRE | AGCTGCGCCGCT | | |
| | LVAGISRS | TCAGGCTTTTCTT | | |
| | GGGTYYP | TCTCCAGCTACC | | |
| | DSVEGRFT | TGATGGCCTGGT | | |
| | ISRDNSKK | TCAGACAAGCAC | | |
| | MVYLQMN | CTGGTAAGGGCA | | |
| | SLRAEGDT | GAGAACTGGTGG | | |
| | AVYYCAR | CTGGCATCAGCA | | |
| | DAGGWGM | GGTCTGGCGGAG | | |
| | DVWGQGT | GGACATACTACC | | |
| | LVTVSS | CCGATTCCGTGG | | |
| | | AGGGACGGTTTA | | |
| | | CTATTTCAAGGG | | |
| | | ACAACTCTAAGA | | |
| | | AAATGGTATACC | | |
| | | TTCAGATGAATA | | |
| | | GTTTGAGGGCAG | | |
| | | AGGGAGATACGG | | |
| | | CCGTGTATTACT | | |
| | | GTGCCAGGGACG | | |
| | | CTGGCGGGTGGG | | |
| | | GCATGGATGTTT | | |
| | | GGGGCCAGGGA | | |
| | | ACCCTGGTCACC | | |
| | | GTCTCCTCA | | |
| SEQ ID | 243 | 244 | | |
| NO: | | | | |
| AMR-02- | EVQLVESG | GAGGTTCAGTTG | No VL | No VL |
| H09 | GGLVQPG | GTCGAGTCTGGT | | |
| | GSLRLSCA | GGTGGGCTCGTG | | |
| | ASGFSFSS | CAACCCGGAGGC | | |
| | YLMAWFR | TCCCTGAGGTTG | | |
| | QAPGKGRE | AGCTGCGCCGCT | | |
| | LVAGISGS | TCAGGCTTTTCTT | | |
| | GAVTYYP | TCTCCAGCTACC | | |
| | DSVEGRFT | TGATGGCCTGGT | | |
| | ISRDNSKK | TCAGACAAGCAC | | |
| | MVYLQMN | CTGGTAAGGGCA | | |
| | SLRAEGDT | GAGAACTGGTGG | | |
| | AVYYCAR | CTGGCATCTCCG | | |
| | DAGGWGM | GGAGCGGAGCCG | | |
| | DVWGQGT | TGACCTACTACC | | |
| | LVTVSS | CCGATTCCGTGG | | |
| | | AGGGACGGTTTA | | |
| | | CTATTTCAAGGG | | |
| | | ACAACTCTAAGA | | |
| | | AAATGGTATACC | | |
| | | TTCAGATGAATA | | |
| | | GTTTGAGGGCAG | | |
| | | AGGGAGATACGG | | |
| | | CCGTGTATTACT | | |
| | | GTGCCAGGGACG | | |
| | | CTGGCGGGTGGG | | |
| | | GCATGGATGTTT | | |
| | | GGGGCCAGGGA | | |
| | | ACCCTGGTCACC | | |
| | | GTCTCCTCA | | |
| SEQ ID | 261 | 262 | | |
| NO: | | | | |
| AMR-02- | EVQLVESG | GAGGTTCAGTTG | No VL | No VL |
| D07 | GGLVQPG | GTCGAGTCTGGT | | |
| | GSLRLSCA | GGTGGGCTCGTG | | |
| | ASGFTFSN | CAACCCGGAGGC | | |
| | YAMSWFR | TCCCTGAGGTTG | | |
| | QAPGKGRE | AGCTGCGCCGCT | | |
| | LVASITGS | TCAGGCTTCACC | | |
| | GATTFYPD | TTTAGCAACTAC | | |
| | SVEGRFTIS | GCCATGTCCTGG | | |
| | RDNAEKL | TTCAGACAAGCA | | |
| | VYLQMNS | CCTGGTAAGGGC | | |
| | LRAEGDTA | AGAGAACTGGTG | | |
| | VYYCAKG | GCTTCCATCACA | | |
| | HGDWYGS | GGGTCTGGCGCT | | |
| | GIDIWGQG | ACTACCTTCTAC | | |
| | TLVTVSS | CCCGATTCCGTG | | |
| | | GAGGGACGGTTT | | |
| | | ACTATTTCAAGG | | |
| | | GACAACGCTGAG | | |
| | | AAATTGGTATAC | | |
| | | CTTCAGATGAAT | | |
| | | AGTTTGAGGGCA | | |
| | | GAGGGAGATACG | | |
| | | GCCGTGTATTAC | | |
| | | TGTGCTAAGGGT | | |
| | | CACGGAGACTGG | | |
| | | TATGGCTCCGGC | | |
| | | ATCGATATTTGG | | |
| | | GGCCAGGGAACC | | |
| | | CTGGTCACCGTC | | |
| | | TCCTCA | | |
| SEQ ID | 279 | 280 | | |
| NO: | | | | |
| AMR-02- | EVQLVESG | GAGGTTCAGTTG | No VL | No VL |
| H08 | GGLVQPG | GTCGAGTCTGGT | | |
| | GSLRLSCA | GGTGGGCTCGTG | | |
| | ASGFTFND | CAACCCGGAGGC | | |
| | YAMSWFR | TCCCTGAGGTTG | | |
| | QAPGKGRE | AGCTGCGCCGCT | | |
| | LVARISGS | TCAGGCTTTACC | | |
| | GGTTNYPD | TTCAACGACTAC | | |
| | SVEGRFTIS | GCCATGAGCTGG | | |
| | RDNPKKLV | TTCAGACAAGCA | | |
| | YLQMNSL | CCTGGTAAGGGC | | |
| | RAEGDTA | AGAGAACTGGTG | | |
| | VYYCARE | GCTAGGATCTCC | | |
| | RLGYPWTF | GGCAGCGGAGG | | |
| | DVWGQGT | GACAACCAACTA | | |
| | LVTVSS | CCCCGATTCCGT | | |
| | | GGAGGGACGGTT | | |
| | | TACTATTTCAAG | | |
| | | GGACAACCCTAA | | |
| | | GAAATTGGTATA | | |
| | | CCTTCAGATGAA | | |
| | | TAGTTTGAGGGC | | |
| | | AGAGGGAGATAC | | |
| | | GGCCGTGTATTA | | |
| | | CTGTGCCAGGGA | | |
| | | GCGGCTGGGGTA | | |
| | | CCCTTGGACCTT | | |
| | | CGACGTGTGGGG | | |
| | | CCAGGGAACCCT | | |
| | | GGTCACCGTCTC | | |
| | | CTCA | | |
| SEQ ID | 297 | 298 | | |
| NO: | | | | |

Exemplary scFv Antibodies.

In some embodiments, the disclosure provides for scFv antibodies. The scFv may also be a tandem scFv, which may be connected by a linker, e.g. a flexible linker. The scFvs on each antibody arm may be connected by a linker, e.g. a flexible linker. An exemplary linker comprises the following amino acid sequence: SGG, GGG, GGS, SSG, SSS, or multiples or combinations thereof. The skilled artisan will understand that framework regions can be modified, as such, the present disclosure includes sequences that have at least 91, 92, 93, 94, 95, 96, 97, 98, 99, or 100 percent sequence identity to the sequences herein.

Table 3 provides exemplary scFv amino acid and nucleic acid sequences.

| | Full | | VH | |
|---|---|---|---|---|
| | Sequence | Full | Sequence | VH |
| AbID | AA | Sequence NT | AA | Sequence NT |
| AMR- | EVQLLES | GAGGTGCAGCTCTTAG | EVQLLESG | GAGGTGCAGCT |
| 02- | GGGLVQP | AAAGCGGAGGTGGCTT | GGLVQPGG | CTTAGAAAGCG |
| G04 | GGSLRLS | GGTTCAGCCTGGCGGT | SLRLSCAAS | GAGGTGGCTTG |
| | CAASGFT | AGTCTAAGACTGTCCT | GFTFERYA | GTTCAGCCTGG |
| | FERYAMT | GTGCAGCTTCTGGCTTC | MTWVRQAP | CGGTAGTCTAA |
| | WVRQAPG | ACTTTTGAACGCTATGC | GKGLEWVS | GACTGTCCTGT |
| | KGLEWVS | CATGACCTGGGTCCGT | GIYANAAG | GCAGCTTCTGG |
| | GIYANAA | CAAGCACCAGGTAAGG | TDYADSVK | CTTCACTTTTG |
| | GTDYADS | GTCTGGAATGGGTGTC | GRFTISRDN | AACGCTATGCC |
| | VKGRFTIS | TGGTATCTACGCCAAC | SKNTLYLQ | ATGACCTGGGT |
| | RDNSKNT | GCAGCGGGCACCGATT | MNSLRAED | CCGTCAAGCAC |
| | LYLQMNS | ATGCCGATAGCGTGAA | TAVYYCAK | CAGGTAAGGGT |
| | LRAEDTA | AGGACGCTTCACCATC | DQSILGIYG | CTGGAATGGGT |
| | VYYCAKD | AGCAGGGACAATTCCA | NAMDYWG | GTCTGGTATCT |
| | QSILGIYG | AGAACACGCTGTACCT | QGTLVTVSS | ACGCCAACGCA |
| | NAMDYW | GCAAATGAACAGCCTG | | GCGGGCACCGA |
| | GQGTLVT | CGAGCCGAGGATACGG | | TTATGCCGATA |
| | VSSGGGG | CCGTGTATTACTGTGCT | | GCGTGAAAGG |
| | SGGGGSG | AAAGATCAAAGCATCC | | ACGCTTCACCA |
| | GGGSDIQ | TGGGAATCTATGGCAA | | TCAGCAGGGAC |
| | MTQSPSSL | CGCGATGGACTATTGG | | AATTCCAAGAA |
| | SASVGDR | GGCCAGGGAACCCTGG | | CACGCTGTACC |
| | VTITCRAS | TCACCGTCTCCTCAGGT | | TGCAAATGAAC |
| | QSVGHYL | GGCGGTGGATCAGGAG | | AGCCTGCGAGC |
| | NWYQQK | GTGGAGGATCTGGAGG | | CGAGGATACGG |
| | PGKAPKL | AGGTGGCAGCGATATT | | CCGTGTATTAC |
| | LIYTSSNL | CAGATGACCCAGAGCC | | TGTGCTAAAGA |
| | QRGVPSR | CATCCTCCCTGTCAGCT | | TCAAAGCATCC |
| | FSGSGSGT | TCTGTAGGAGACCGCG | | TGGGAATCTAT |
| | DFTLTISS | TTACCATCACCTGTCGT | | GGCAACGCGAT |
| | LQPEDFA | GCCTCCCAGAGCGTGG | | GGACTATTGGG |
| | TYYCQQS | GCCACTACCTGAACTG | | GCCAGGGAACC |
| | HSSPWTF | GTATCAGCAGAAGCCT | | CTGGTCACCGT |
| | GGGTKVE | GGTAAGGCACCGAAGC | | CTCCTCA |
| | IK | TGCTGATCTATACCTCC | | |
| | | TCTAACCTGCAACGTG | | |
| | | GCGTCCCGTCACGCTTT | | |
| | | AGTGGCTCTGGTTCTGG | | |
| | | GACCGACTTCACTCTCA | | |
| | | CCATTAGCAGTCTGCA | | |
| | | ACCGGAGGACTTCGCG | | |
| | | ACATACTATTGCCAGC | | |
| | | AGAGCCACTCTTCCCC | | |
| | | GTGGACTTTTGGAGGC | | |
| | | GGTACCAAGGTGGAGA | | |
| | | TCAAA | | |
| SEQ ID | 1 | 2 | 3 | 4 |
| NO: | | | | |
| AMR- | EVQLLES | GAGGTGCAGCTCTTAG | EVQLLESG | GAGGTGCAGCT |
| 02- | GGGLVQP | AAAGCGGAGGTGGCTT | GGLVQPGG | CTTAGAAAGCG |
| E01 | GGSLRLS | GGTTCAGCCTGGCGGT | SLRLSCAAS | GAGGTGGCTTG |
| | CAASGFT | AGTCTAAGACTGTCCT | GFTFERYA | GTTCAGCCTGG |
| | FERYAMT | GTGCAGCTTCTGGCTTC | MTWVRQAP | CGGTAGTCTAA |
| | WVRQAPG | ACTTTTGAACGCTATGC | GKGLEWVS | GACTGTCCTGT |
| | KGLEWVS | CATGACCTGGGTCCGT | GIYANAAG | GCAGCTTCTGG |
| | GIYANAA | CAAGCACCAGGTAAGG | TDYADSVK | CTTCACTTTTG |
| | GTDYADS | GTCTGGAATGGGTGTC | GRFTISRDN | AACGCTATGCC |
| | VKGRFTIS | TGGTATCTACGCCAAC | SKNTLYLQ | ATGACCTGGGT |
| | RDNSKNT | GCAGCGGGCACCGATT | MNSLRAED | CCGTCAAGCAC |
| | LYLQMNS | ATGCCGATAGCGTGAA | TAVYYCAK | CAGGTAAGGGT |
| | LRAEDTA | AGGACGCTTCACCATC | DQSILGIYG | CTGGAATGGGT |
| | VYYCAKD | AGCAGGGACAATTCCA | NAMDYWG | GTCTGGTATCT |
| | QSILGIYG | AGAACACGCTGTACCT | QGTLVTVSS | ACGCCAACGCA |
| | NAMDYW | GCAAATGAACAGCCTG | | GCGGGCACCGA |
| | GQGTLVT | CGAGCCGAGGATACGG | | TTATGCCGATA |
| | VSSGGGG | CCGTGTATTACTGTGCT | | GCGTGAAAGG |
| | SGGGGSG | AAAGATCAAAGCATCC | | ACGCTTCACCA |
| | GGGSDIQ | TGGGAATCTATGGCAA | | TCAGCAGGGAC |
| | MTQSPSSL | CGCGATGGACTATTGG | | AATTCCAAGAA |
| | SASVGDR | GGCCAGGGAACCCTGG | | CACGCTGTACC |
| | VTITCRAS | TCACCGTCTCCTCAGGT | | TGCAAATGAAC |
| | QSVGHYL | GGCGGTGGATCAGGAG | | AGCCTGCGAGC |
| | NWYQQK | GTGGAGGATCTGGAGG | | CGAGGATACGG |
| | PGKAPKL | AGGTGGCAGCGATATT | | CCGTGTATTAC |
| | LIYATSTL | CAGATGACCCAGAGCC | | TGTGCTAAAGA |
| | HSGVPSRF | CATCCTCCCTGTCAGCT | | TCAAAGCATCC |
| | SGSGSGT | TCTGTAGGAGACCGCG | | TGGGAATCTAT |
| | DFTLTISS | TTACCATCACCTGTCGT | | GGCAACGCGAT |
| | LQPEDFA | GCCTCCCAGAGCGTGG | | GGACTATTGGG |
| | TYYCQQS | GCCACTACCTGAACTG | | GCCAGGGAACC |
| | YGNPRTF | GTATCAGCAGAAGCCT | | CTGGTCACCGT |
| | GGGTKVE | GGTAAGGCACCGAAGC | | CTCCTCA |
| | IK | TGCTGATCTATGCCACC | | |
| | | AGCACTCTGCACTCTG | | |
| | | GCGTCCCGTCACGCTTT | | |
| | | AGTGGCTCTGGTTCTGG | | |
| | | GACCGACTTCACTCTCA | | |
| | | CCATTAGCAGTCTGCA | | |
| | | ACCGGAGGACTTCGCG | | |
| | | ACATACTATTGCCAGC | | |
| | | AGAGCTATGGTAATCC | | |
| | | GCGCACCTTTGGAGGC | | |
| | | GGTACCAAGGTGGAGA | | |
| | | TCAAA | | |
| SEQ ID | 35 | 36 | 37 | 38 |
| NO: | | | | |
| AMR- | EVQLLES | GAGGTGCAGCTCTTAG | EVQLLESG | GAGGTGCAGCT |
| 01- | GGGLVQP | AAAGCGGAGGTGGCTT | GGLVQPGG | CTTAGAAAGCG |
| C11 | GGSLRLS | GGTTCAGCCTGGCGGT | SLRLSCAAS | GAGGTGGCTTG |
| | CAASGFT | AGTCTAAGACTGTCCT | GFTFSSYGL | GTTCAGCCTGG |
| | FSSYGLA | GTGCAGCTTCTGGTTTT | AWVRQAPG | CGGTAGTCTAA |
| | WVRQAPG | ACCTTCAGCTCTTATGG | KGLEWVSA | GACTGTCCTGT |
| | KGLEWVS | CCTGGCTTGGGTCCGTC | ISGSGVNAH | GCAGCTTCTGG |
| | AISGSGVN | AAGCACCAGGTAAGGG | YADSVKGR | TTTTACCTTCA |
| | AHYADSV | TCTGGAATGGGTGTCT | FTISRDNSK | GCTCTTATGGC |
| | KGRFTISR | GCAATCTCTGGCTCCG | NTLYLQMN | CTGGCTTGGGT |
| | DNSKNTL | GTGTGAACGCTCACTA | SLRAEDTA | CCGTCAAGCAC |
| | YLQMNSL | TGCCGATAGCGTGAAA | VYYCAKDQ | CAGGTAAGGGT |
| | RAEDTAV | GGACGCTTCACCATCA | SILGIYGNA | CTGGAATGGGT |
| | YYCAKDQ | GCAGGGACAATTCCAA | MDYWGQG | GTCTGCAATCT |
| | SILGIYGN | GAACACGCTGTACCTG | TLVTVSS | CTGGCTCCGGT |
| | AMDYWG | CAAATGAACAGCCTGC | | GTGAACGCTCA |
| | QGTLVTV | GAGCCGAGGATACGGC | | CTATGCCGATA |
| | SSGGGGS | CGTGTATTACTGTGCTA | | GCGTGAAAGG |
| | GGGGSGG | AAGATCAAAGCATCCT | | ACGCTTCACCA |
| | GGSDIQM | GGGAATCTATGGCAAC | | TCAGCAGGGAC |
| | TQSPSSLS | GCGATGGACTATTGGG | | AATTCCAAGAA |
| | ASVGDRV | GCCAGGGAACCCTGGT | | CACGCTGTACC |
| | TITCRASQ | CACCGTCTCCTCAGGTG | | TGCAAATGAAC |
| | SVGHYLN | GCGGTGGATCAGGAGG | | AGCCTGCGAGC |
| | WYQQKP | TGGAGGATCTGGAGGA | | CGAGGATACGG |
| | GKAPKLLI | GGTGGCAGCGATATTC | | CCGTGTATTAC |
| | YTSSNLQ | AGATGACCCAGAGCCC | | TGTGCTAAAGA |
| | RGVPSRFS | ATCCTCCCTGTCAGCTT | | TCAAAGCATCC |
| | GSGSGTD | CTGTAGGAGACCGCGT | | TGGGAATCTAT |
| | FTLTISSL | TACCATCACCTGTCGTG | | GGCAACGCGAT |
| | QPEDFAT | CCTCCCAGAGCGTGGG | | GGACTATTGGG |
| | YYCQQSH | CCACTACCTGAACTGG | | GCCAGGGAACC |
| | SSPWTFG | TATCAGCAGAAGCCTG | | CTGGTCACCGT |
| | GGTKVEI | GTAAGGCACCGAAGCT | | CTCCTCA |
| | K | GCTGATCTATACCTCCT | | |
| | | CTAACCTGCAACGTGG | | |
| | | CGTCCCGTCACGCTTTA | | |
| | | GTGGCTCTGGTTCTGGG | | |
| | | ACCGACTTCACTCTCAC | | |
| | | CATTAGCAGTCTGCAA | | |
| | | CCGGAGGACTTCGCGA | | |
| | | CATACTATTGCCAGCA | | |
| | | GAGCCACTCTTCCCCGT | | |
| | | GGACTTTTGGAGGCGG | | |
| | | TACCAAGGTGGAGATC | | |
| | | AAA | | |
| SEQ ID | 69 | 70 | 7 1 | 72 |
| NO: | | | | |
| AMR- | EVQLLES | GAGGTGCAGCTCTTAG | EVQLLESG | GAGGTGCAGCT |
| 01- | GGGLVQP | AAAGCGGAGGTGGCTT | GGLVQPGG | CTTAGAAAGCG |
| B12 | GGSLRLS | GGTTCAGCCTGGCGGT | SLRLSCAAS | GAGGTGGCTTG |
| | CAASGLS | AGTCTAAGACTGTCCT | GLSFSSHA | GTTCAGCCTGG |
| | FSSHAMS | GTGCAGCTTCTGGCCTG | MSWVRQAP | CGGTAGTCTAA |
| | WVRQAPG | TCTTTTAGCAGCCACGC | GKGLEWVS | GACTGTCCTGT |
| | KGLEWVS | TATGTCATGGGTCCGTC | GLSASGGR | GCAGCTTCTGG |
| | GLSASGG | AAGCACCAGGTAAGGG | TYYADSVK | CCTGTCTTTTA |
| | RTYYADS | TCTGGAATGGGTGTCT | GRFTISRDN | GCAGCCACGCT |
| | VKGRFTIS | GGTCTGTCTGCAAGCG | SKNTLYLQ | ATGTCATGGGT |
| | RDNSKNT | GAGGCCGCACCTACTA | MNSLRAED | CCGTCAAGCAC |
| | LYLQMNS | TGCCGATAGCGTGAAA | TAVYYCAR | CAGGTAAGGGT |
| | LRAEDTA | GGACGCTTCACCATCA | DRGRYGAE | CTGGAATGGGT |
| | VYYCARD | GCAGGGACAATTCCAA | SAYPVRAF | GTCTGGTCTGT |
| | RGRYGAE | GAACACGCTGTACCTG | DPWGQGTL | CTGCAAGCGGA |
| | SAYPVRA | CAAATGAACAGCCTGC | VTVSS | GGCCGCACCTA |
| | FDPWGQG | GAGCCGAGGATACGGC | | CTATGCCGATA |
| | TLVTVSS | CGTGTATTACTGTGCCA | | GCGTGAAAGG |
| | GGGGSGG | GGGATCGGGGTAGGTA | | ACGCTTCACCA |
| | GGSGGGG | CGGGGCCGAATCCGCG | | TCAGCAGGGAC |
| | SDIQMTQ | TATCCCGTGCGGGCCTT | | AATTCCAAGAA |
| | SPSSLSAS | TGACCCTTGGGGCCAG | | CACGCTGTACC |
| | VGDRVTI | GGAACCCTGGTCACCG | | TGCAAATGAAC |
| | TCRASQSI | TCTCCTCAGGTGGCGGT | | AGCCTGCGAGC |
| | SSYLNWY | GGATCAGGAGGTGGAG | | CGAGGATACGG |
| | QQKPGKA | GATCTGGAGGAGGTGG | | CCGTGTATTAC |
| | PKLLIYAT | CAGCGATATTCAGATG | | TGTGCCAGGGA |
| | STLHSGVP | ACCCAGAGCCCATCCT | | TCGGGGTAGGT |
| | SRFSGSGS | CCCTGTCAGCTTCTGTA | | ACGGGGCCGA |
| | GTDFTLTI | GGAGACCGCGTTACCA | | ATCCGCGTATC |
| | SSLQPEDF | TCACCTGTCGTGCCTCC | | CCGTGCGGGCC |
| | ATYYCQQ | CAATCTATTAGCAGCT | | TTTGACCCTTG |
| | SYGNPRT | ACCTGAACTGGTATCA | | GGGCCAGGGA |
| | FGGGTKV | GCAGAAGCCTGGTAAG | | ACCCTGGTCAC |
| | EIK | GCACCGAAGCTGCTGA | | CGTCTCCTCA |
| | | TCTATGCCACCAGCACT | | |
| | | CTGCACTCTGGCGTCCC | | |
| | | GTCACGCTTTAGTGGCT | | |
| | | CTGGTTCTGGGACCGA | | |
| | | CTTCACTCTCACCATTA | | |
| | | GCAGTCTGCAACCGGA | | |
| | | GGACTTCGCGACATAC | | |
| | | TATTGCCAGCAGAGCT | | |
| | | ATGGTAATCCGCGCAC | | |
| | | CTTTGGAGGCGGTACC | | |
| | | AAGGTGGAGATCAAA | | |
| SEQ ID | 103 | 104 | 105 | 106 |
| NO: | | | | |
| AMR- | EVQLLES | GAGGTGCAGCTCTTAG | EVQLLESG | GAGGTGCAGCT |
| 01- | GGGLVQP | AAAGCGGAGGTGGCTT | GGLVQPGG | CTTAGAAAGCG |
| C10 | GGSLRLS | GGTTCAGCCTGGCGGT | SLRLSCAAS | GAGGTGGCTTG |
| | CAASGFT | AGTCTAAGACTGTCCT | GFTFERYA | GTTCAGCCTGG |
| | FERYAMT | GTGCAGCTTCTGGCTTC | MTWVRQAP | CGGTAGTCTAA |
| | WVRQAPG | ACTTTTGAACGCTATGC | GKGLEWVS | GACTGTCCTGT |
| | KGLEWVS | CATGACCTGGGTCCGT | GIYANAAG | GCAGCTTCTGG |
| | GIYANAA | CAAGCACCAGGTAAGG | TDYADSVK | CTTCACTTTTG |
| | GTDYADS | GTCTGGAATGGGTGTC | GRFTISRDN | AACGCTATGCC |
| | VKGRFTIS | TGGTATCTACGCCAAC | SKNTLYLQ | ATGACCTGGGT |
| | RDNSKNT | GCAGCGGGCACCGATT | MNSLRAED | CCGTCAAGCAC |
| | LYLQMNS | ATGCCGATAGCGTGAA | TAVYYCAK | CAGGTAAGGGT |
| | LRAEDTA | AGGACGCTTCACCATC | DQSILGIYG | CTGGAATGGGT |
| | VYYCAKD | AGCAGGGACAATTCCA | NAMDYWG | GTCTGGTATCT |
| | QSILGIYG | AGAACACGCTGTACCT | QGTLVTVSS | ACGCCAACGCA |
| | NAMDYW | GCAAATGAACAGCCTG | | GCGGGCACCGA |
| | GQGTLVT | CGAGCCGAGGATACGG | | TTATGCCGATA |
| | VSSGGGG | CCGTGTATTACTGTGCT | | GCGTGAAAGG |
| | SGGGGSG | AAAGATCAAAGCATCC | | ACGCTTCACCA |
| | GGGSDIQ | TGGGAATCTATGGCAA | | TCAGCAGGGAC |
| | MTQSPSSL | CGCGATGGACTATTGG | | AATTCCAAGAA |
| | SASVGDR | GGCCAGGGAACCCTGG | | CACGCTGTACC |
| | VTITCRAS | TCACCGTCTCCTCAGGT | | TGCAAATGAAC |
| | QPISVYLH | GGCGGTGGATCAGGAG | | AGCCTGCGAGC |
| | WYQQKP | GTGGAGGATCTGGAGG | | CGAGGATACGG |
| | GKAPKLLI | AGGTGGCAGCGATATT | | CCGTGTATTAC |
| | YAASSLQ | CAGATGACCCAGAGCC | | TGTGCTAAAGA |
| | SGVPSRFS | CATCCTCCCTGTCAGCT | | TCAAAGCATCC |
| | GSGSGTD | TCTGTAGGAGACCGCG | | TGGGAATCTAT |
| | FTLTISSL | TTACCATCACCTGTCGT | | GGCAACGCGAT |
| | QPEDFAT | GCCTCCCAGCCGATTA | | GGACTATTGGG |
| | YYCQQTY | GCGTGTACCTGCACTG | | GCCAGGGAACC |
| | STPHTFGG | GTATCAGCAGAAGCCT | | CTGGTCACCGT |
| | GTKVEIK | GGTAAGGCACCGAAGC | | CTCCTCA |
| | | TGCTGATCTATGCCGCA | | |
| | | TCCAGCCTCCAGTCTGG | | |
| | | CGTCCCGTCACGCTTTA | | |
| | | GTGGCTCTGGTTCTGGG | | |
| | | ACCGACTTCACTCTCAC | | |
| | | CATTAGCAGTCTGCAA | | |
| | | CCGGAGGACTTCGCGA | | |
| | | CATACTATTGCCAGCA | | |
| | | GACCTATAGCACCCCG | | |
| | | CATACTTTTGGAGGCG | | |
| | | GTACCAAGGTGGAGAT | | |
| | | CAAA | | |
| SEQ ID | 137 | 138 | 139 | 140 |
| NO: | | | | |
| AMR- | EVQLLES | GAGGTGCAGCTCTTAG | EVQLLESG | GAGGTGCAGCT |
| 01- | GGGLVQP | AAAGCGGAGGTGGCTT | GGLVQPGG | CTTAGAAAGCG |
| H09 | GGSLRLS | GGTTCAGCCTGGCGGT | SLRLSCAAS | GAGGTGGCTTG |
| | CAASGFS | AGTCTAAGACTGTCCT | GFSLRNYA | GTTCAGCCTGG |
| | LRNYAVS | GTGCAGCTTCTGGCTTT | VSWVRQAP | CGGTAGTCTAA |
| | WVRQAPG | AGCTTGCGCAACTATG | GKGLEWVS | GACTGTCCTGT |
| | KGLEWVS | CTGTGTCCTGGGTCCGT | TISGGGGRT | GCAGCTTCTGG |
| | TISGGGGR | CAAGCACCAGGTAAGG | HYADSVKG | CTTTAGCTTGC |
| | THYADSV | GTCTGGAATGGGTGTC | RFTISRDNS | GCAACTATGCT |
| | KGRFTISR | TACCATCTCCGGTGGC | KNTLYLQM | GTGTCCTGGGT |
| | DNSKNTL | GGTGGCCGCACTCACT | NSLRAEDT | CCGTCAAGCAC |
| | YLQMNSL | ATGCCGATAGCGTGAA | AVYYCART | CAGGTAAGGGT |
| | RAEDTAV | AGGACGCTTCACCATC | GPGIVGRVS | CTGGAATGGGT |
| | YYCARTG | AGCAGGGACAATTCCA | GAFDVWGQ | GTCTACCATCT |
| | PGIVGRVS | AGAACACGCTGTACCT | GTLVTVSS | CCGGTGGCGGT |
| | GAFDVW | GCAAATGAACAGCCTG | | GGCCGCACTCA |
| | GQGTLVT | CGAGCCGAGGATACGG | | CTATGCCGATA |
| | VSSGGGG | CCGTGTATTACTGTGCA | | GCGTGAAAGG |
| | SGGGGSG | CGTACTGGCCCTGGGA | | ACGCTTCACCA |
| | GGGSDIQ | TCGTCGGACGGGTTTC | | TCAGCAGGGAC |
| | MTQSPSSL | AGGCGCTTTTGATGTCT | | AATTCCAAGAA |
| | SASVGDR | GGGGCCAGGGAACCCT | | CACGCTGTACC |
| | VTITCRTS | GGTCACCGTCTCCTCAG | | TGCAAATGAAC |
| | QSITKYLN | GTGGCGGTGGATCAGG | | AGCCTGCGAGC |
| | WYQQKP | AGGTGGAGGATCTGGA | | CGAGGATACGG |
| | GKAPKLLI | GGAGGTGGCAGCGATA | | CCGTGTATTAC |
| | YSASSLQS | TTCAGATGACCCAGAG | | TGTGCACGTAC |
| | GVPSRFSG | CCCATCCTCCCTGTCAG | | TGGCCCTGGGA |
| | SGSGTDFT | CTTCTGTAGGAGACCG | | TCGTCGGACGG |
| | LTISSLQP | CGTTACCATCACCTGTC | | GTTTCAGGCGC |
| | EDFATYY | GTACATCCCAGAGCAT | | TTTTGATGTCT |
| | CQQSYSA | CACTAAATACCTGAAC | | GGGGCCAGGG |
| | PYSFGGG | TGGTATCAGCAGAAGC | | AACCCTGGTCA |
| | TKVEIK | CTGGTAAGGCACCGAA | | CCGTCTCCTCA |
| | | GCTGCTGATCTATTCTG | | |
| | | CCTCCAGCCTGCAAAG | | |
| | | CGGCGTCCCGTCACGC | | |
| | | TTTAGTGGCTCTGGTTC | | |
| | | TGGGACCGACTTCACT | | |
| | | CTCACCATTAGCAGTCT | | |
| | | GCAACCGGAGGACTTC | | |
| | | GCGACATACTATTGCC | | |
| | | AGCAGTCTTACTCCGC | | |
| | | ACCGTATAGCTTTGGA | | |
| | | GGCGGTACCAAGGTGG | | |
| | | AGATCAAA | | |
| SEQ ID | 171 | 172 | 173 | 174 |
| NO: | | | | |

TABLE 4
Additional Sequences.

| SEQ ID NO: | | |
|---|---|---|
| 305 | CTRTM_VH | EVQLVESGGGLVKPGGSLKLSCAASGFTFSDYYMFWVRQ |
| | | TPEKRLEWVATISDGGSYTYYPGGVKGRFTISRDNAKNNL |
| | | YLQMTSLKSEDTAMYFCARDGGMLTTRAMDYWGQGTSV |
| | | TVSS |
| 306 | Peptide + | KCNTATCATQRLANFLVHSSNNFGPILPPTNVGSNTYGSGS |
| | linker | ATGGSGSGASSGSGSATGS |
| 307 | CTRTM VL | DIQMTQTTSSLSASLGDRVTISCSASQGISNYLNWYQQKPD |
| | | GTVKLLIYYTSSLHSGVPSRFSGSGSGTDYSLTISNLEPEDIA |
| | | TYYCQHYSEFPFTFGSGTKLEIK |
| 308 | Peptide-HC- | KCNTATCATQRLANFLVHSSNNFGPILPPTNVGSNTYGSGS |
| | CTRTM | ATGGSGSGASSGSGSATGSEVQLVESGGGLVKPGGSLKLS |
| | | CAASGFTFSDYYMFWVRQTPEKRLEWVATISDGGSYTYY |
| | | PGGVKGRFTISRDNAKNNLYLQMTSLKSEDTAMYFCARD |
| | | GGMLTTRAMDYWGQGTSVTVSSASTKGPSVFPLAPCSRST |
| | | SESTAALGCLVKDYFPEPVTVSWNSGALTSGVHTFPAVLQ |
| | | SSGLYSLSSVVTVPSSSLGTKTYTCNVDHKPSNTKVDKRV |
| | | ESKYGPPCPPCPAPEFLGGPSVFLFPPKPKDTLMISRTPEVT |
| | | CVVVDVSQEDPEVQFNWYVDGVEVHNAKTKPREEQFNST |
| | | YRVVSVLTVLHQDWLNGKEYKCKVSNKGLPSSIEKTISKA |
| | | KGQPREPQVYTLPPSQEEMTKNQVSLTCLVKGFYPSDIAV |
| | | EWESNGQPENNYKTTPPVLDSDGSFFLYSRLTVDKSRWQE |
| | | GNVFSCSVMHEALHNHYTQKSLSLSLG |
| 309 | LC-CTRTM | DIQMTQTTSSLSASLGDRVTISCSASQGISNYLNWYQQKPD |
| | | GTVKLLIYYTSSLHSGVPSRFSGSGSGTDYSLTISNLEPEDIA |
| | | TYYCQHYSEFPFTFGSGTKLEIKRTVAAPSVFIFPPSDEQLK |
| | | SGTASVVCLLNNFYPREAKVQWKVDNALQSGNSQESVTE |
| | | QDSKDSTYSLSSTLTLSKADYEKHKVYACEVTHQGLSSPV |
| | | TKSFNRGEC |
| 310 | CTRTM_VH_DNA | GAGGTGCAGCTGGTGGAAAGCGGCGGCGGCCTGGTGAA |
| | | GCCTGGCGGCAGCCTGAAGCTGAGCTGCGCCGCCTCCG |
| | | GCTTCACCTTCTCTGACTACTACATGTTCTGGGTTAGGC |
| | | AGACCCCCGAGAAGAGGCTGGAGTGGGTGGCCACCATC |
| | | AGCGACGGCGGCAGCTACACCTACTACCCCGGCGGCGT |
| | | TAAGGGCAGGTTCACCATCAGCAGGGACAACGCCAAGA |
| | | ACAACCTGTACCTGCAGATGACCAGCCTGAAAAGCGAG |
| | | GACACCGCCATGTACTTTTGCGCCAGGGACGGCGGCAT |
| | | GCTGACCACACGCGCCATGGACTACTGGGGCCAGGGCA |
| | | CCAGCGTGACCGTGAGCAGC |
| 311 | Peptide + | AAGTGCAATACCGCTACCTGCGCTACCCAGAGGCTCGCT |
| | linker_DNA | AACTTCCTGGTGCACAGCTCTAACAATTTCGGCCCCATC |
| | | CTGCCCCCCACCAACGTGGGAAGCAACACCTACGGAAG |
| | | CGGCAGTGCCACCGGCGGCTCTGGGAGCGGCGCCTCCT |
| | | CTGGAAGCGGCTCTGCCACCGGCAGC |
| 312 | CTRTM_VL_DNA | GACATCCAGATGACCCAGACCACCAGCAGCCTGAGCGC |
| | | CTCCCTGGGCGACAGGGTGACCATCAGCTGCAGCGCCA |
| | | GCCAGGGCATCAGCAACTACCTGAACTGGTACCAGCAG |
| | | AAGCCCGACGGCACCGTGAAGCTGCTGATCTACTACAC |
| | | CTCCAGCCTGCACAGCGGCGTGCCCAGCAGGTTCAGCG |
| | | GCAGCGGAAGCGGCACCGACTACAGCCTGACCATCAGC |
| | | AACCTCGAACCCGAGGACATCGCCACCTACTACTGCCA |
| | | GCACTACAGCGAGTTCCCCTTCACCTTCGGCAGCGGCAC |
| | | CAAGCTGGAGATCAAG |
| 313 | Peptide-HC- | AAGTGCAATACCGCTACCTGCGCTACCCAGAGGCTCGCT |
| | CTRTM_DNA | AACTTCCTGGTGCACAGCTCTAACAATTTCGGCCCCATC |
| | | CTGCCCCCCACCAACGTGGGAAGCAACACCTACGGAAG |
| | | CGGCAGTGCCACCGGCGGCTCTGGGAGCGGCGCCTCCT |
| | | CTGGAAGCGGCTCTGCCACCGGCAGCGAGGTGCAGCTG |
| | | GTGGAAAGCGGCGGCGGCCTGGTGAAGCCTGGCGGCAG |
| | | CCTGAAGCTGAGCTGCGCCGCCTCCGGCTTCACCTTCTC |
| | | TGACTACTACATGTTCTGGGTTAGGCAGACCCCCGAGAA |
| | | GAGGCTGGAGTGGGTGGCCACCATCAGCGACGGCGGCA |
| | | GCTACACCTACTACCCCGGCGGCGTTAAGGGCAGGTTC |
| | | ACCATCAGCAGGGACAACGCCAAGAACAACCTGTACCT |
| | | GCAGATGACCAGCCTGAAAAGCGAGGACACCGCCATGT |
| | | ACTTTTGCGCCAGGGACGGCGGCATGCTGACCACACGC |
| | | GCCATGGACTACTGGGGCCAGGGCACCAGCGTGACCGT |
| | | GAGCAGCgcatccaccaagggcccatccgtcttccccctggcgccctgctccaggag |
| | | cacctccgagagcacagccgccctgggctgcctggtcaaggactacttccccgaaccggtg |
| | | acggtgtcgtggaactcaggcgccctgaccagcggcgtgcacaccttcccggctgtcctac |
| | | agtcctcaggactctactccctcagcagcgtggtgaccgtgccctccagcagcttgggcacg |
| | | aagacctacacctgcaacgtagatcacaagcccagcaacaccaaggtggacaagagagtt |
| | | gagtccaaatatggtcccccatgcccaccttgcccagcacctgagttcctggggggaccatc |
| | | agtcttcctgttccccccaaaacccaaggacactctcatgatctcccggacccctgaggtcac |
| | | gtgcgtggtggtggacgtgagccaggaagaccccgaggtccagttcaactggtacgtggat |
| | | ggcgtggaggtgcataatgccaagacaaagccgcgggaggagcagttcaacagcacgta |
| | | ccgtgtggtcagcgtcctcaccgtcctgcaccaggactggctgaacggcaaggagtacaag |
| | | tgcaaggtgtccaacaaaggcctcccgtcctccatcgagaaaaccatctccaaagccaaag |
| | | ggcagccccgagagccacaggtgtacaccctgcccccatcccaggaggagatgaccaag |
| | | aaccaggtcagcctgacctgcctggtcaaaggcttctaccccagcgacatcgccgtggagt |
| | | gggagagcaatgggcagccggagaacaactacaagaccacgcctcccgtgctggactcc |
| | | gacggctccttcttcctctacagcaggctaaccgtggacaagagcaggtggcaggagggga |
| | | atgtcttctcatgctccgtgatgcatgaggctctgcacaaccactacacacagaagagcctctc |
| | | cctgtctctgggt |
| 314 | LC- | GACATCCAGATGACCCAGACCACCAGCAGCCTGAGCGC |
| | CTRTM_DNA | CTCCCTGGGCGACAGGGTGACCATCAGCTGCAGCGCCA |
| | | GCCAGGGCATCAGCAACTACCTGAACTGGTACCAGCAG |
| | | AAGCCCGACGGCACCGTGAAGCTGCTGATCTACTACAC |
| | | CTCCAGCCTGCACAGCGGCGTGCCCAGCAGGTTCAGCG |
| | | GCAGCGGAAGCGGCACCGACTACAGCCTGACCATCAGC |
| | | AACCTCGAACCCGAGGACATCGCCACCTACTACTGCCA |
| | | GCACTACAGCGAGTTCCCCTTCACCTTCGGCAGCGGCAC |
| | | CAAGCTGGAGATCAAGCGAACTGTGGCTGCACCATCTG |
| | | TCTTCATCTTCCCGCCATCTGATGAGCAGTTGAAATCTG |
| | | GAACTGCCTCTGTTGTGTGCCTGCTGAATAACTTCTATC |
| | | CCAGAGAGGCCAAAGTACAGTGGAAGGTGGATAACGCC |
| | | CTCCAATCGGGTAACTCCCAGGAGAGTGTCACAGAGCA |
| | | GGACAGCAAGGACAGCACCTACAGCCTCAGCAGCACCC |
| | | TGACGCTGAGCAAAGCAGACTACGAGAAACACAAAGTC |
| | | TACGCCTGCGAAGTCACCCATCAGGGCCTGAGCTCGCCC |
| | | GTCACAAAGAGCTTCAACAGGGGAGAGTGT |
| 315 | CTRTM | DYYMF |
| | hCDR1 | |
| 316 | CTRTM | TISDGGSYTYYPGGVKG |
| | hCDR2 | |
| 317 | CTRTM | DGGMLTTRAMDY |
| | hCDR3 | |
| 318 | CTRTM | SASQGISNYLN |
| | lCDR1 | |
| 319 | CTRTM | YTSSLHS |
| | lCDR2 | |
| 320 | CTRTM | QHYSEFPFT |
| | lCDR3 | |

The present disclosure also includes methods of treating a subject with a therapeutic agent that treats or inhibits a metabolic disorder comprising administering or continuing to administer to the subject the therapeutic agent that treats or inhibits the metabolic disorder in a standard dosage amount, and administering to the subject an anti-amylin receptor antibody, binding fragments, or nanobody.

The present disclosure also includes methods of treating a subject with a therapeutic agent that treats or inhibits a cardiovascular disease, wherein the subject is suffering from a cardiovascular disease, comprising administering or continuing to administer to the subject the therapeutic agent that treats or inhibits the cardiovascular disease in a standard dosage amount, and administering to the subject an anti-amylin receptor antibody, binding fragments, or nanobody.

For subjects that have an increased risk of developing a metabolic disorder, such as type 2 diabetes, lipodystrophy, liver inflammation, fatty liver disease, hypercholesterolemia, elevated liver enzymes (such as, for example, ALT and/or AST), obesity, high blood pressure, and/or elevated triglyceride level (hypertriglyceridemia), and/or a cardiovascular disease, such as cardiomyopathy, heart failure, and high blood pressure. These subjects can be treated with an anti-amylin receptor antibody, binding fragments, or nanobody of the present disclosure.

In certain embodiments of the disclosure, the metabolic disorder is type 2 diabetes, obesity, NASH, and/or elevated triglyceride level. In any of the embodiments disclosed herein, the metabolic disorder is type 2 diabetes. In any of the embodiments disclosed herein, the metabolic disorder is obesity. In any of the embodiments disclosed herein, the metabolic disorder is NASH. In any of the embodiments disclosed herein, the metabolic disorder is elevated triglyceride level. In any of the embodiments disclosed herein, the metabolic disorder is lipodystrophy. In any of the embodiments disclosed herein, the metabolic disorder is liver inflammation. In any of the embodiments disclosed herein, the metabolic disorder is fatty liver disease. In any of the embodiments disclosed herein, the metabolic disorder is hypercholesterolemia. In any of the embodiments disclosed herein, the metabolic disorder is elevated liver enzymes (such as, for example, ALT and/or AST).

In addition, other metabolic disorders/conditions associated with body fat distribution also include, but are not limited to: type 2 diabetes, hyperlipidemia or dyslipidemia (high or altered circulating levels of low-density lipoprotein cholesterol (LDL-C), triglycerides, very low-density lipoprotein cholesterol (VLDL-C), apolipoprotein B or other lipid fractions), obesity (particularly abdominal obesity), lipodystrophy (such as an inability to deposit fat in adipose depots regionally (partial lipodystrophy) or in the whole body (lipoatrophy)), insulin resistance or higher or altered insulin levels at fasting or during a glucose or insulin challenge, liver fat deposition or fatty liver disease and their complications (such as, for example, cirrhosis, fibrosis, or inflammation of the liver), higher or elevated or altered liver enzyme levels or other markers of liver damage, inflammation or fat deposition, higher blood pressure and/or hypertension, higher blood sugar or glucose or hyperglycemia, metabolic syndrome, coronary artery disease, and other atherosclerotic conditions, and the complications of each of the aforementioned conditions.

### Administration of Therapeutic an Anti-Amylin Receptor Antibody, Binding Fragments, or Nanobody.

The in vivo administration of the therapeutic an anti-amylin receptor antibody, binding fragments, or nanobody described herein may be carried out intravenously, intramuscularly, subcutaneously, topically, orally, transdermally, intraperitoneally, intraorbitally, intrathecally, intraventricularly, intranasally, transmucosally, through implantation, or through inhalation. Intravenous administration may be carried out via injection or infusion. In some embodiments, the Anti-Activin E antibodies of the disclosure are administered intravenously. In some embodiments, the Anti-Activin E antibodies of the disclosure are administered subcutaneously. Administration of the therapeutic Anti-Activin E antibodies may be performed with any suitable excipients, carriers, or Binding Activity.

### Binding Activity.

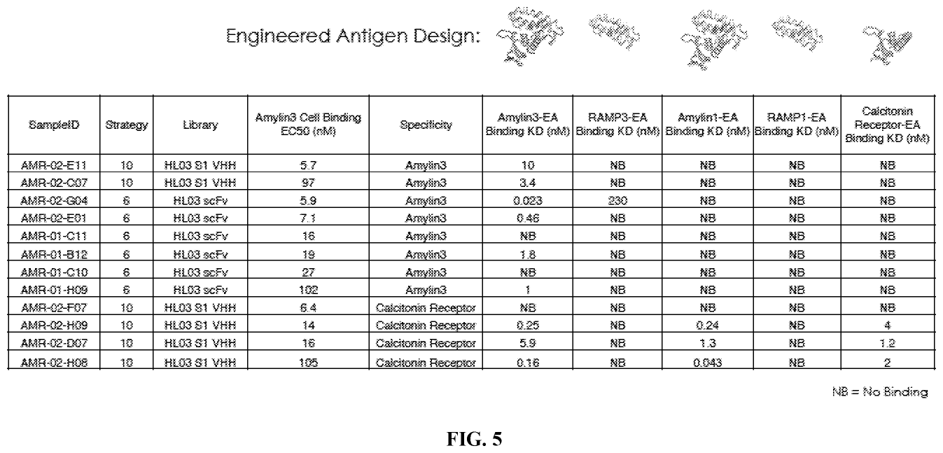

The following table includes a comparison of the different binding epitopes of the antibodies and nanobodies disclosed herein. FIG. 5 shows the results from Naïve scFv and VHH libraries yield Amylin3 receptor specific cell binders, also summarized as Table 5.

TABLE 5

| | | Amylin3 | | | | RAMP1- | CALCR- |
|---|---|---|---|---|---|---|---|
| | | Cell | Amylin3- | RAMP3- | Amylin1- | EA | EA |
| | | Binding | EA | EA | EA | Binding | Binding |
| Antibody | | EC50 | Binding | Binding | Binding | KD | KD |
| ID | Library | (nM) | KD (nM) | KD (nM) | KD (nM) | (nM) | (nM) |
| AMR- | HL03 | 5.9 | 0.023 | 230 | NB | NB | NB |
| 02-G04 | scFv | | | | | | |
| AMR- | HL03 | 7.1 | 0.46 | NB | NB | NB | NB |
| 02-E01 | scFv | | | | | | |
| AMR- | HL03 | 16 | NB | NB | NB | NB | NB |
| 01-C11 | scFv | | | | | | |
| AMR- | HL03 | 19 | 1.8 | NB | NB | NB | NE |
| 01-B12 | scFv | | | | | | |
| AMR- | HL03 | 27 | NB | NB | NB | NB | NE |
| 01-C10 | scFv | | | | | | |
| AMR- | HL03 | 102 | 1 | NB | NB | NB | NB |
| 01-H09 | scFv | | | | | | |
| AMR- | HL03 | 5.7 | 10 | NB | NB | NB | NB |
| 02-E11 | S1 | | | | | | |
| | VHH | | | | | | |
| AMR- | HL03 | 97 | 3.4 | NB | NB | NB | NE |
| 02-C07 | S1 | | | | | | |
| | VHH | | | | | | |
| AMR- | HL03 | 6.4 | NB | NB | NB | NB | NB |
| 02-F07 | S1 | | | | | | |
| | VHH | | | | | | |
| AMR- | HL03 | 14 | 0.25 | NB | 0.24 | NB | 4 |
| 02-H09 | S1 | | | | | | |
| | VHH | | | | | | |
| AMR- | HL03 | 16 | 5.9 | NB | 1.3 | NB | 1.2 |
| 02-D07 | S1 | | | | | | |
| | VHH | | | | | | |
| AMR- | HL03 | 105 | 0.16 | NB | 0.043 | NB | 2 |
| 02-H08 | S1 | | | | | | |
| | VHH | | | | | | |

NB: no binding.

### Methods for Acute Food Intake.

Male C57BL/6J mice (10 per group), aged 18 weeks and maintained on a high-fat diet (HFD; 60% kcal from fat) for 12 weeks to induce obesity, were used to evaluate the effect of test compounds on acute food intake. Animals were randomized into three treatment groups (n=10 per group): Group 1 received vehicle (PBS), Group 2 received Peptide-HC-CTRTM (10 mg/kg), and Group 3 received cagrilintide (30 nmol/kg). Cagrilintide is a long-acting analogue of amylin (PubChem—167312356).

On the day of the study, mice were dosed subcutaneously with vehicle or test article. Immediately following dosing, all animals were fasted for 4 hours. After the fasting period, pre-weighed food was returned to the cages, and food intake was measured at 3 hours and again at 6 hours post-refeeding. Food consumption was determined by weighing remaining food and accounting for spillage. Intake values were recorded in grams per mouse. All procedures were conducted in accordance with institutional animal care guidelines.

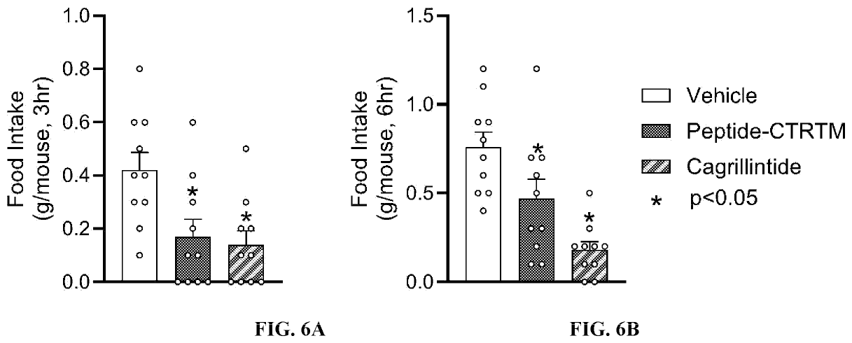

FIGS. 6A and 6B show the effect of test articles on acute food intake, measured at 3 hours (FIG. 6A) and 6 hours (FIG. 6B) post-feeding. Dosing was performed 4 hours prior to feeding.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

For each of the claims, each dependent claim can depend both from the independent claim and from each of the prior dependent claims for each and every claim so long as the prior claim provides a proper antecedent basis for a claim term or element.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. An anti-amylin receptor antibody, antigen-binding fragments thereof, or nanobody, wherein the antibody, antigen-binding fragments thereof, or nanobody comprises:
a heavy chain variable domain (VH) complementarity determining region (CDR) 1, VH CDR2, and VH CDR3 comprising an amino acid sequence of SEQ ID NOS: 8, 10, 12; 42, 44, 46; 76, 78, 80; 110, 112, 114; 144, 146, 148; 178, 180, 182; or 315, 316, 317, respectively; and
a light chain variable domain (VL) CDR1, VL CDR2, VL CDR3 comprising an amino acid sequence of SEQ ID NOs: 22, 24, 26; 56, 58, 60; 90, 92, 94; 124, 126, 128; 158, 160, 162; 192, 194, 196; or 318, 319, 320, respectively; or
a VHH CDR1, CDR2, CDR3 comprising an amino acid sequence of SEQ ID NOs: 210, 212, 214; 228, 230, 232; 246, 248, 250; 264, 266, 268; 282, 284, 286; or 300, 302, 304.

2. The antibody, fragments thereof, or nanobody of claim 1, wherein the antibody or nanobody, comprises:
a VH comprising an amino acid having at least 90, 91, 92, 93, 94, 95, 96, 97, 98, 99, or 100% sequence identity to SEQ ID NOS: 3, 37, 71, 105, 139, 173, or 305 and
a VL comprising the amino acid sequence of any one of the following SEQ ID NOs: an amino acid having at least 90, 91, 92, 93, 94, 95, 96, 97, 98, 99, or 100% sequence identity to SEQ ID NOS: 5, 39, 73, 107, 141, 175, or 307;
a VHH comprising an amino acid having at least 90, 91, 92, 93, 94, 95, 96, 97, 98, 99, or 100% sequence identity to SEQ ID NOS: 210, 228, 246, 264, 282, or 300; or
is an ScFv having an amino acid having at least 90, 91, 92, 93, 94, 95, 96, 97, 98, 99, or 100% sequence identity to SEQ ID NO: 1, 35, 69, 103, 137, or 171.

3. The antibody, fragments thereof, or nanobody of claim 1, wherein the antibody is a monoclonal antibody.

4. The antibody, fragments thereof, or nanobody of claim 1, wherein the antibody is a full-length antibody.

5. The antibody, fragments thereof, or nanobody of claim 1, wherein the antibody is an antibody fragment selected from Fab, Fab′, F(ab′)₂, Fv, single-chain variable fragment (scFv), or VHH.

6. The antibody, fragments thereof, or nanobody of claim 5, wherein the antibody or nanobody is fused to an Fc domain of any one of the following: human IgG1, human IgG2, human IgG3, and human IgG4.

7. A method of treating a disease in a subject in need thereof, comprising administering to the subject a therapeutically effective amount of an antibody, antigen-binding fragments thereof, or a nanobody comprising:
a heavy chain variable domain (VH) complementarity determining region (CDR) 1, VH CDR2, and VH CDR3 comprising an amino acid sequence of SEQ ID NOS: 8, 10, 12; 42, 44, 46; 76, 78, 80; 110, 112, 114; 144, 146, 148; 178, 180, 182; or 315, 316, 317, respectively; and
a light chain variable domain (VL) CDR1, VL CDR2, VL CDR3 comprising an amino acid sequence of SEQ ID NOs: 22, 24, 26; 56, 58, 60; 90, 92, 94; 124, 126, 128; 158, 160, 162; 192, 194, 196; or 318, 319, 320, respectively; or
a VHH CDR1, CDR2, CDR3 comprising an amino acid sequence of SEQ ID NOs: 210, 212, 214; 228, 230, 232; 246, 248, 250; 264, 266, 268; 282, 284, 286; or 300, 302, 304.

8. The method of claim 7, where the disease is obesity.

9. The method of claim 7, where the disease is selected from at least one of: a metabolic disorder, type 2 diabetes, an elevated triglyceride level, lipodystrophy, liver inflammation, fatty liver disease, hypercholesterolemia, an elevated liver enzyme, nonalcoholic steatohepatitis (NASH), a cardiovascular disease, cardiomyopathy, high blood pressure, or heart failure.

10. The method of claim 7, wherein the subject is human.

11. A scFv anti-amylin receptor antibody comprising:
a heavy chain variable domain (VH) complementarity determining region (CDR) 1, VH CDR2, and VH CDR3 comprising an amino acid sequence of SEQ ID NOS: 8, 10, 12; 42, 44, 46; 76, 78, 80; 110, 112, 114; 144, 146, 148; or 178, 180, 182; or 315, 316, 317, respectively; and
a light chain variable domain (VL) CDR1, VL CDR2, VL CDR3 comprising an amino acid sequence of SEQ ID NOs: 22, 24, 26; 56, 58, 60; 90, 92, 94; 124, 126, 128; 158, 160, 162; 192, 194, 196; or 318, 319, 320, respectively.

12. The antibody of claim 11, wherein the antibody has a total of two or four scFv binding sites in a single scFv-Fc formatted antibody.

13. The antibody of claim 12, wherein an scFv1 of each antibody arm comprises a first heavy chain variable domain (VH1) and a first light chain variable domain (VL1); and wherein an scFv2 of each antibody arm comprises a first heavy chain variable domain (VH2) and a first light chain variable domain (VL2).

14. The antibody of claim 13, wherein the antibody or antigen-binding fragment thereof comprises: at least 90, 91, 92, 93, 94, 95, 96, 97, 98, 99, or 100% sequence identity to SEQ ID NOS: 1, 35, 69, 103, 137, or 171.

15. The antibody of claim 14, wherein a linker comprises the following amino acid sequence: SGG, GGG, GGS, SSG, SSS, or multiples or combinations thereof.

16. A method of treating a disease in a subject in need thereof, comprising administering to the subject a therapeutically effective amount of the antibody comprising:
a heavy chain variable domain (VH) complementarity determining region (CDR) 1, VH CDR2, and VH CDR3 comprising an amino acid sequence of SEQ ID NOS: 8, 10, 12; 42, 44, 46; 76, 78, 80; 110, 112, 114; 144, 146, 148; or 178, 180, 182; or 315, 316, 317, respectively; and
a light chain variable domain (VL) CDR1, VL CDR2, VL CDR3 comprising an amino acid sequence of SEQ ID NOs: 22, 24, 26; 56, 58, 60; 90, 92, 94; 124, 126, 128; 158, 160, 162; 192, 194, 196; or 318, 319, 320, respectively.

17. The method of claim 16, where the disease is obesity.

18. The method of claim 16, where the disease is selected from at least one of: a metabolic disorder, type 2 diabetes, obesity, an elevated triglyceride level, lipodystrophy, liver inflammation, fatty liver disease, hypercholesterolemia, an elevated liver enzyme, nonalcoholic steatohepatitis (NASH), a cardiovascular disease, cardiomyopathy, high blood pressure, or heart failure.

19. The method of claim 16, wherein the subject is human.

20. A nucleic acid that expresses an anti-amylin receptor antibody, antigen-binding fragments thereof, or nanobody, wherein the antibody, antigen-binding fragments thereof, or a nanobody comprising:
a heavy chain variable domain encoded by a polynucleotide having at least 95, 96, 97, 98, 99, or 100% sequence identity to SEQ ID NOS: 8, 10, 12; 42, 44, 46; 76, 78, 80; 110, 112, 114; 144, 146, 148; or 178, 180, 182; or 310; and
a light chain variable domain encoded by a polynucleotide having at least 95, 96, 97, 98, 99, or 100% sequence identity to SEQ ID NOS: 22, 24, 26; 56, 58, 60; 90, 92, 94; 124, 126, 128; 158, 160, 162; or 192, 194, 196; or 312; or
a VHH encoded by a polynucleotide having at least 95%, 96%, 97%, 98%, 99%, or 100% sequence identity to SEQ ID NOS: 212, 230, 248, 266, 284, or 302.

21. The nucleic acid of claim 20, wherein the antibody is a monoclonal, bispecific, multivalent, multi-specific, diabody, chimeric, scFv antibody, or antigen-binding fragments thereof.

22. The nucleic acid of claim 20, wherein an antigen-binding domain is fused to an Fc domain of any one of the following: human IgG1, human IgG2, human IgG3, and human IgG4.

23. The nucleic acid of claim 19, wherein a nucleic acid sequence is optimized for expression in a bacterial, fungal, mammalian, insect, or plant cell.

24. The nucleic acid of claim 19, wherein the antibody or antigen-binding fragment is encoded by a polynucleotide having at least 95, 96, 97, 98, 99, or 100% sequence identity to SEQ ID NOS; 2, 36, 70, 104, 138, 172, or 313 or 212, 230, 248, 266, 284, 302, or 314.

25. The nucleic acid of claim 19, further comprising a vector.

26. The nucleic acid of claim 25, further comprising a host cell comprising the vector.

27. An antigenic peptide comprising an amino acid having at least 90, 91, 92, 93, 94, 95, 96, 97, 98, 99, or 100% sequence identity to SEQ ID NOS: 206, 308, or 309.

28. The antigenic peptide of claim 27, wherein the peptide is encoded by a polynucleotide having at least 95%, 96%, 97%, 98%, 99%, or 100% sequence identity to SEQ ID NOS: 308, 311, or 313.


SEQUENCE LISTING (322 sequences)

SEQ ID NO: 1 - protein, 245 aa, synthetic construct
EVQLLESGGGLVQPGGSLRLSCAASGFTFERYAMTWVRQAPGKGLEWVSGIYANAAGTDYADSVKGRFTISRDNSKNTLYLQMNSLRAEDTAVYYCAKDQSILGIYGNAMDYWGQGTLVTVSSGGGGSGGGGSGGGGSDIQMTQSPSSLSASVGDRVTITCRASQSVGHYLNWYQQKPGKAPKLLIYTSSNLQRGVPSRFSGSGSGTDFTLTISSLQPEDFATYYCQQSHSSPWTFGGGTKVEIK

SEQ ID NO: 2 - DNA, 735 bp, synthetic construct
  source 1..735: mol_type = other DNA
gaggtgcagctcttagaaagcggaggtggcttggttcagcctggcggtagtctaagactgtcctgtgcagcttctggcttcacttttgaacgctatgccatgacctgggtccgtcaagcaccaggtaagggtctggaatgggtgtctggtatctacgccaacgcagcgggcaccgattatgccgatagcgtgaaaggacgcttcaccatcagcagggacaattccaagaacacgctgtacctgcaaatgaacagcctgcgagccgaggatacggccgtgtattactgtgctaaagatcaaagcatcctgggaatctatggcaacgcgatggactattggggccagggaaccctggtcaccgtctcctcaggtggcggtggatcaggaggtggaggatctggaggaggtggcagcgatattcagatgacccagagcccatcctccctgtcagcttctgtaggagaccgcgttaccatcacctgtcgtgcctcccagagcgtgggccactacctgaactggtatcagcagaagcctggtaaggcaccgaagctgctgatctatacctcctctaacctgcaacgtggcgtcccgtcacgctttagtggctctggttctgggaccgacttcactctcaccattagcagtctgcaaccggaggacttcgcgacatactattgccagcagagccactcttccccgtggacttttggaggcggtaccaaggtggagatcaaa

SEQ ID NO: 3 - protein, 123 aa, synthetic construct
EVQLLESGGGLVQPGGSLRLSCAASGFTFERYAMTWVRQAPGKGLEWVSGIYANAAGTDYADSVKGRFTISRDNSKNTLYLQMNSLRAEDTAVYYCAKDQSILGIYGNAMDYWGQGTLVTVSS

SEQ ID NO: 4 - DNA, 369 bp, synthetic construct
  source 1..369: mol_type = other DNA
gaggtgcagctcttagaaagcggaggtggcttggttcagcctggcggtagtctaagactgtcctgtgcagcttctggcttcacttttgaacgctatgccatgacctgggtccgtcaagcaccaggtaagggtctggaatgggtgtctggtatctacgccaacgcagcgggcaccgattatgccgatagcgtgaaaggacgcttcaccatcagcagggacaattccaagaacacgctgtacctgcaaatgaacagcctgcgagccgaggatacggccgtgtattactgtgctaaagatcaaagcatcctgggaatctatggcaacgcgatggactattggggccagggaaccctggtcaccgtctcctca

SEQ ID NO: 5 - protein, 107 aa, synthetic construct
DIQMTQSPSSLSASVGDRVTITCRASQSVGHYLNWYQQKPGKAPKLLIYTSSNLQRGVPSRFSGSGSGTDFTLTISSLQPEDFATYYCQQSHSSPWTFGGGTKVEIK

SEQ ID NO: 6 - DNA, 321 bp, synthetic construct
  source 1..321: mol_type = other DNA
gatattcagatgacccagagcccatcctccctgtcagcttctgtaggagaccgcgttaccatcacctgtcgtgcctcccagagcgtgggccactacctgaactggtatcagcagaagcctggtaaggcaccgaagctgctgatctatacctcctctaacctgcaacgtggcgtcccgtcacgctttagtggctctggttctgggaccgacttcactctcaccattagcagtctgcaaccggaggacttcgcgacatactattgccagcagagccactcttccccgtggacttttggaggcggtaccaaggtggagatcaaa

SEQ ID NO: 7 - skipped

SEQ ID NO: 8 - protein, 8 aa, synthetic construct
GFTFERYA

SEQ ID NO: 9 - skipped

SEQ ID NO: 10 - protein, 8 aa, synthetic construct
IYANAAGT

SEQ ID NO: 11 - skipped

SEQ ID NO: 12 - protein, 16 aa, synthetic construct
AKDQSILGIYGNAMDY

SEQ ID NO: 13-21 - skipped (9 entries)

SEQ ID NO: 22 - protein, 6 aa, synthetic construct
QSVGHY

SEQ ID NO: 23-25 - skipped (3 entries)

SEQ ID NO: 26 - protein, 9 aa, synthetic construct
QQSHSSPWT

SEQ ID NO: 27-34 - skipped (8 entries)

SEQ ID NO: 35 - protein, 245 aa, synthetic construct
EVQLLESGGGLVQPGGSLRLSCAASGFTFERYAMTWVRQAPGKGLEWVSGIYANAAGTDYADSVKGRFTISRDNSKNTLYLQMNSLRAEDTAVYYCAKDQSILGIYGNAMDYWGQGTLVTVSSGGGGSGGGGSGGGGSDIQMTQSPSSLSASVGDRVTITCRASQSVGHYLNWYQQKPGKAPKLLIYATSTLHSGVPSRFSGSGSGTDFTLTISSLQPEDFATYYCQQSYGNPRTFGGGTKVEIK

SEQ ID NO: 36 - DNA, 735 bp, synthetic construct
  source 1..735: mol_type = other DNA
gaggtgcagctcttagaaagcggaggtggcttggttcagcctggcggtagtctaagactgtcctgtgcagcttctggcttcacttttgaacgctatgccatgacctgggtccgtcaagcaccaggtaagggtctggaatgggtgtctggtatctacgccaacgcagcgggcaccgattatgccgatagcgtgaaaggacgcttcaccatcagcagggacaattccaagaacacgctgtacctgcaaatgaacagcctgcgagccgaggatacggccgtgtattactgtgctaaagatcaaagcatcctgggaatctatggcaacgcgatggactattggggccagggaaccctggtcaccgtctcctcaggtggcggtggatcaggaggtggaggatctggaggaggtggcagcgatattcagatgacccagagcccatcctccctgtcagcttctgtaggagaccgcgttaccatcacctgtcgtgcctcccagagcgtgggccactacctgaactggtatcagcagaagcctggtaaggcaccgaagctgctgatctatgccaccagcactctgcactctggcgtcccgtcacgctttagtggctctggttctgggaccgacttcactctcaccattagcagtctgcaaccggaggacttcgcgacatactattgccagcagagctatggtaatccgcgcacctttggaggcggtaccaaggtggagatcaaa

SEQ ID NO: 37 - protein, 123 aa, synthetic construct; identical to SEQ ID NO: 3

SEQ ID NO: 38 - DNA, 369 bp, synthetic construct; identical to SEQ ID NO: 4
  source 1..369: mol_type = other DNA

SEQ ID NO: 39 - protein, 107 aa, synthetic construct
DIQMTQSPSSLSASVGDRVTITCRASQSVGHYLNWYQQKPGKAPKLLIYATSTLHSGVPSRFSGSGSGTDFTLTISSLQPEDFATYYCQQSYGNPRTFGGGTKVEIK

SEQ ID NO: 40 - DNA, 321 bp, synthetic construct
  source 1..321: mol_type = other DNA
gatattcagatgacccagagcccatcctccctgtcagcttctgtaggagaccgcgttaccatcacctgtcgtgcctcccagagcgtgggccactacctgaactggtatcagcagaagcctggtaaggcaccgaagctgctgatctatgccaccagcactctgcactctggcgtcccgtcacgctttagtggctctggttctgggaccgacttcactctcaccattagcagtctgcaaccggaggacttcgcgacatactattgccagcagagctatggtaatccgcgcacctttggaggcggtaccaaggtggagatcaaa

SEQ ID NO: 41 - skipped

SEQ ID NO: 42 - protein, 8 aa, synthetic construct
GFTFERYA

SEQ ID NO: 43 - skipped

SEQ ID NO: 44 - protein, 8 aa, synthetic construct
IYANAAGT

SEQ ID NO: 45 - skipped

SEQ ID NO: 46 - protein, 16 aa, synthetic construct
AKDQSILGIYGNAMDY

SEQ ID NO: 47-55 - skipped (9 entries)

SEQ ID NO: 56 - protein, 6 aa, synthetic construct
QSVGHY

SEQ ID NO: 57-59 - skipped (3 entries)

SEQ ID NO: 60 - protein, 9 aa, synthetic construct
QQSYGNPRT

SEQ ID NO: 61-68 - skipped (8 entries)

SEQ ID NO: 69 - protein, 245 aa, synthetic construct
EVQLLESGGGLVQPGGSLRLSCAASGFTFSSYGLAWVRQAPGKGLEWVSAISGSGVNAHYADSVKGRFTISRDNSKNTLYLQMNSLRAEDTAVYYCAKDQSILGIYGNAMDYWGQGTLVTVSSGGGGSGGGGSGGGGSDIQMTQSPSSLSASVGDRVTITCRASQSVGHYLNWYQQKPGKAPKLLIYTSSNLQRGVPSRFSGSGSGTDFTLTISSLQPEDFATYYCQQSHSSPWTFGGGTKVEIK

SEQ ID NO: 70 - DNA, 735 bp, synthetic construct
  source 1..735: mol_type = other DNA
gaggtgcagctcttagaaagcggaggtggcttggttcagcctggcggtagtctaagactgtcctgtgcagcttctggttttaccttcagctcttatggcctggcttgggtccgtcaagcaccaggtaagggtctggaatgggtgtctgcaatctctggctccggtgtgaacgctcactatgccgatagcgtgaaaggacgcttcaccatcagcagggacaattccaagaacacgctgtacctgcaaatgaacagcctgcgagccgaggatacggccgtgtattactgtgctaaagatcaaagcatcctgggaatctatggcaacgcgatggactattggggccagggaaccctggtcaccgtctcctcaggtggcggtggatcaggaggtggaggatctggaggaggtggcagcgatattcagatgacccagagcccatcctccctgtcagcttctgtaggagaccgcgttaccatcacctgtcgtgcctcccagagcgtgggccactacctgaactggtatcagcagaagcctggtaaggcaccgaagctgctgatctatacctcctctaacctgcaacgtggcgtcccgtcacgctttagtggctctggttctgggaccgacttcactctcaccattagcagtctgcaaccggaggacttcgcgacatactattgccagcagagccactcttccccgtggacttttggaggcggtaccaaggtggagatcaaa

SEQ ID NO: 71 - protein, 123 aa, synthetic construct
EVQLLESGGGLVQPGGSLRLSCAASGFTFSSYGLAWVRQAPGKGLEWVSAISGSGVNAHYADSVKGRFTISRDNSKNTLYLQMNSLRAEDTAVYYCAKDQSILGIYGNAMDYWGQGTLVTVSS

SEQ ID NO: 72 - DNA, 369 bp, synthetic construct
  source 1..369: mol_type = other DNA
gaggtgcagctcttagaaagcggaggtggcttggttcagcctggcggtagtctaagactgtcctgtgcagcttctggttttaccttcagctcttatggcctggcttgggtccgtcaagcaccaggtaagggtctggaatgggtgtctgcaatctctggctccggtgtgaacgctcactatgccgatagcgtgaaaggacgcttcaccatcagcagggacaattccaagaacacgctgtacctgcaaatgaacagcctgcgagccgaggatacggccgtgtattactgtgctaaagatcaaagcatcctgggaatctatggcaacgcgatggactattggggccagggaaccctggtcaccgtctcctca

SEQ ID NO: 73 - protein, 107 aa, synthetic construct; identical to SEQ ID NO: 5

SEQ ID NO: 74 - DNA, 321 bp, synthetic construct; identical to SEQ ID NO: 6
  source 1..321: mol_type = other DNA

SEQ ID NO: 75 - skipped

SEQ ID NO: 76 - protein, 8 aa, synthetic construct
GFTFSSYG

SEQ ID NO: 77 - skipped

SEQ ID NO: 78 - protein, 8 aa, synthetic construct
ISGSGVNA

SEQ ID NO: 79 - skipped

SEQ ID NO: 80 - protein, 16 aa, synthetic construct
AKDQSILGIYGNAMDY

SEQ ID NO: 81-89 - skipped (9 entries)

SEQ ID NO: 90 - protein, 6 aa, synthetic construct
QSVGHY

SEQ ID NO: 91-93 - skipped (3 entries)

SEQ ID NO: 94 - protein, 9 aa, synthetic construct
QQSHSSPWT

SEQ ID NO: 95-102 - skipped (8 entries)

SEQ ID NO: 103 - protein, 249 aa, synthetic construct
EVQLLESGGGLVQPGGSLRLSCAASGLSFSSHAMSWVRQAPGKGLEWVSGLSASGGRTYYADSVKGRFTISRDNSKNTLYLQMNSLRAEDTAVYYCARDRGRYGAESAYPVRAFDPWGQGTLVTVSSGGGGSGGGGSGGGGSDIQMTQSPSSLSASVGDRVTITCRASQSISSYLNWYQQKPGKAPKLLIYATSTLHSGVPSRFSGSGSGTDFTLTISSLQPEDFATYYCQQSYGNPRTFGGGTKVEIK

SEQ ID NO: 104 - DNA, 747 bp, synthetic construct
  source 1..747: mol_type = other DNA
gaggtgcagctcttagaaagcggaggtggcttggttcagcctggcggtagtctaagactgtcctgtgcagcttctggcctgtcttttagcagccacgctatgtcatgggtccgtcaagcaccaggtaagggtctggaatgggtgtctggtctgtctgcaagcggaggccgcacctactatgccgatagcgtgaaaggacgcttcaccatcagcagggacaattccaagaacacgctgtacctgcaaatgaacagcctgcgagccgaggatacggccgtgtattactgtgccagggatcggggtaggtacggggccgaatccgcgtatcccgtgcgggcctttgacccttggggccagggaaccctggtcaccgtctcctcaggtggcggtggatcaggaggtggaggatctggaggaggtggcagcgatattcagatgacccagagcccatcctccctgtcagcttctgtaggagaccgcgttaccatcacctgtcgtgcctcccaatctattagcagctacctgaactggtatcagcagaagcctggtaaggcaccgaagctgctgatctatgccaccagcactctgcactctggcgtcccgtcacgctttagtggctctggttctgggaccgacttcactctcaccattagcagtctgcaaccggaggacttcgcgacatactattgccagcagagctatggtaatccgcgcacctttggaggcggtaccaaggtggagatcaaa

SEQ ID NO: 105 - protein, 127 aa, synthetic construct
EVQLLESGGGLVQPGGSLRLSCAASGLSFSSHAMSWVRQAPGKGLEWVSGLSASGGRTYYADSVKGRFTISRDNSKNTLYLQMNSLRAEDTAVYYCARDRGRYGAESAYPVRAFDPWGQGTLVTVSS

SEQ ID NO: 106 - DNA, 381 bp, synthetic construct
  source 1..381: mol_type = other DNA
gaggtgcagctcttagaaagcggaggtggcttggttcagcctggcggtagtctaagactgtcctgtgcagcttctggcctgtcttttagcagccacgctatgtcatgggtccgtcaagcaccaggtaagggtctggaatgggtgtctggtctgtctgcaagcggaggccgcacctactatgccgatagcgtgaaaggacgcttcaccatcagcagggacaattccaagaacacgctgtacctgcaaatgaacagcctgcgagccgaggatacggccgtgtattactgtgccagggatcggggtaggtacggggccgaatccgcgtatcccgtgcgggcctttgacccttggggccagggaaccctggtcaccgtctcctca

SEQ ID NO: 107 - protein, 107 aa, synthetic construct
DIQMTQSPSSLSASVGDRVTITCRASQSISSYLNWYQQKPGKAPKLLIYATSTLHSGVPSRFSGSGSGTDFTLTISSLQPEDFATYYCQQSYGNPRTFGGGTKVEIK

SEQ ID NO: 108 - DNA, 321 bp, synthetic construct
  source 1..321: mol_type = other DNA
gatattcagatgacccagagcccatcctccctgtcagcttctgtaggagaccgcgttaccatcacctgtcgtgcctcccaatctattagcagctacctgaactggtatcagcagaagcctggtaaggcaccgaagctgctgatctatgccaccagcactctgcactctggcgtcccgtcacgctttagtggctctggttctgggaccgacttcactctcaccattagcagtctgcaaccggaggacttcgcgacatactattgccagcagagctatggtaatccgcgcacctttggaggcggtaccaaggtggagatcaaa

SEQ ID NO: 109 - skipped

SEQ ID NO: 110 - protein, 8 aa, synthetic construct
GLSFSSHA

SEQ ID NO: 111 - skipped

SEQ ID NO: 112 - protein, 8 aa, synthetic construct
LSASGGRT

SEQ ID NO: 113 - skipped

SEQ ID NO: 114 - protein, 20 aa, synthetic construct
ARDRGRYGAESAYPVRAFDP

SEQ ID NO: 115-123 - skipped (9 entries)

SEQ ID NO: 124 - protein, 6 aa, synthetic construct
QSISSY

SEQ ID NO: 125-127 - skipped (3 entries)

SEQ ID NO: 128 - protein, 9 aa, synthetic construct
QQSYGNPRT

SEQ ID NO: 129-136 - skipped (8 entries)

SEQ ID NO: 137 - protein, 245 aa, synthetic construct
EVQLLESGGGLVQPGGSLRLSCAASGFTFERYAMTWVRQAPGKGLEWVSGIYANAAGTDYADSVKGRFTISRDNSKNTLYLQMNSLRAEDTAVYYCAKDQSILGIYGNAMDYWGQGTLVTVSSGGGGSGGGGSGGGGSDIQMTQSPSSLSASVGDRVTITCRASQPISVYLHWYQQKPGKAPKLLIYAASSLQSGVPSRFSGSGSGTDFTLTISSLQPEDFATYYCQQTYSTPHTFGGGTKVEIK

SEQ ID NO: 138 - DNA, 735 bp, synthetic construct
  source 1..735: mol_type = other DNA
gaggtgcagctcttagaaagcggaggtggcttggttcagcctggcggtagtctaagactgtcctgtgcagcttctggcttcacttttgaacgctatgccatgacctgggtccgtcaagcaccaggtaagggtctggaatgggtgtctggtatctacgccaacgcagcgggcaccgattatgccgatagcgtgaaaggacgcttcaccatcagcagggacaattccaagaacacgctgtacctgcaaatgaacagcctgcgagccgaggatacggccgtgtattactgtgctaaagatcaaagcatcctgggaatctatggcaacgcgatggactattggggccagggaaccctggtcaccgtctcctcaggtggcggtggatcaggaggtggaggatctggaggaggtggcagcgatattcagatgacccagagcccatcctccctgtcagcttctgtaggagaccgcgttaccatcacctgtcgtgcctcccagccgattagcgtgtacctgcactggtatcagcagaagcctggtaaggcaccgaagctgctgatctatgccgcatccagcctccagtctggcgtcccgtcacgctttagtggctctggttctgggaccgacttcactctcaccattagcagtctgcaaccggaggacttcgcgacatactattgccagcagacctatagcaccccgcatacttttggaggcggtaccaaggtggagatcaaa

SEQ ID NO: 139 - protein, 123 aa, synthetic construct; identical to SEQ ID NO: 3

SEQ ID NO: 140 - DNA, 369 bp, synthetic construct; identical to SEQ ID NO: 4
  source 1..369: mol_type = other DNA

SEQ ID NO: 141 - protein, 107 aa, synthetic construct
DIQMTQSPSSLSASVGDRVTITCRASQPISVYLHWYQQKPGKAPKLLIYAASSLQSGVPSRFSGSGSGTDFTLTISSLQPEDFATYYCQQTYSTPHTFGGGTKVEIK

SEQ ID NO: 142 - DNA, 321 bp, synthetic construct
  source 1..321: mol_type = other DNA
gatattcagatgacccagagcccatcctccctgtcagcttctgtaggagaccgcgttaccatcacctgtcgtgcctcccagccgattagcgtgtacctgcactggtatcagcagaagcctggtaaggcaccgaagctgctgatctatgccgcatccagcctccagtctggcgtcccgtcacgctttagtggctctggttctgggaccgacttcactctcaccattagcagtctgcaaccggaggacttcgcgacatactattgccagcagacctatagcaccccgcatacttttggaggcggtaccaaggtggagatcaaa

SEQ ID NO: 143 - skipped

SEQ ID NO: 144 - protein, 8 aa, synthetic construct
GFTFERYA

SEQ ID NO: 145 - skipped

SEQ ID NO: 146 - protein, 8 aa, synthetic construct
IYANAAGT

SEQ ID NO: 147 - skipped

SEQ ID NO: 148 - protein, 16 aa, synthetic construct
AKDQSILGIYGNAMDY

SEQ ID NO: 149-157 - skipped (9 entries)

SEQ ID NO: 158 - protein, 6 aa, synthetic construct
QPISVY

SEQ ID NO: 159-161 - skipped (3 entries)

SEQ ID NO: 162 - protein, 9 aa, synthetic construct
QQTYSTPHT

SEQ ID NO: 163-170 - skipped (8 entries)

SEQ ID NO: 171 - protein, 246 aa, synthetic construct
EVQLLESGGGLVQPGGSLRLSCAASGFSLRNYAVSWVRQAPGKGLEWVSTISGGGGRTHYADSVKGRFTISRDNSKNTLYLQMNSLRAEDTAVYYCARTGPGIVGRVSGAFDVWGQGTLVTVSSGGGGSGGGGSGGGGSDIQMTQSPSSLSASVGDRVTITCRTSQSITKYLNWYQQKPGKAPKLLIYSASSLQSGVPSRFSGSGSGTDFTLTISSLQPEDFATYYCQQSYSAPYSFGGGTKVEIK

SEQ ID NO: 172 - DNA, 738 bp, synthetic construct
  source 1..738: mol_type = other DNA
gaggtgcagctcttagaaagcggaggtggcttggttcagcctggcggtagtctaagactgtcctgtgcagcttctggctttagcttgcgcaactatgctgtgtcctgggtccgtcaagcaccaggtaagggtctggaatgggtgtctaccatctccggtggcggtggccgcactcactatgccgatagcgtgaaaggacgcttcaccatcagcagggacaattccaagaacacgctgtacctgcaaatgaacagcctgcgagccgaggatacggccgtgtattactgtgcacgtactggccctgggatcgtcggacgggtttcaggcgcttttgatgtctggggccagggaaccctggtcaccgtctcctcaggtggcggtggatcaggaggtggaggatctggaggaggtggcagcgatattcagatgacccagagcccatcctccctgtcagcttctgtaggagaccgcgttaccatcacctgtcgtacatcccagagcatcactaaatacctgaactggtatcagcagaagcctggtaaggcaccgaagctgctgatctattctgcctccagcctgcaaagcggcgtcccgtcacgctttagtggctctggttctgggaccgacttcactctcaccattagcagtctgcaaccggaggacttcgcgacatactattgccagcagtcttactccgcaccgtatagctttggaggcggtaccaaggtggagatcaaa

SEQ ID NO: 173 - protein, 124 aa, synthetic construct
EVQLLESGGGLVQPGGSLRLSCAASGFSLRNYAVSWVRQAPGKGLEWVSTISGGGGRTHYADSVKGRFTISRDNSKNTLYLQMNSLRAEDTAVYYCARTGPGIVGRVSGAFDVWGQGTLVTVSS

SEQ ID NO: 174 - DNA, 372 bp, synthetic construct
  source 1..372: mol_type = other DNA
gaggtgcagctcttagaaagcggaggtggcttggttcagcctggcggtagtctaagactgtcctgtgcagcttctggctttagcttgcgcaactatgctgtgtcctgggtccgtcaagcaccaggtaagggtctggaatgggtgtctaccatctccggtggcggtggccgcactcactatgccgatagcgtgaaaggacgcttcaccatcagcagggacaattccaagaacacgctgtacctgcaaatgaacagcctgcgagccgaggatacggccgtgtattactgtgcacgtactggccctgggatcgtcggacgggtttcaggcgcttttgatgtctggggccagggaaccctggtcaccgtctcctca

SEQ ID NO: 175 - protein, 107 aa, synthetic construct
DIQMTQSPSSLSASVGDRVTITCRTSQSITKYLNWYQQKPGKAPKLLIYSASSLQSGVPSRFSGSGSGTDFTLTISSLQPEDFATYYCQQSYSAPYSFGGGTKVEIK

SEQ ID NO: 176 - DNA, 321 bp, synthetic construct
  source 1..321: mol_type = other DNA
gatattcagatgacccagagcccatcctccctgtcagcttctgtaggagaccgcgttaccatcacctgtcgtacatcccagagcatcactaaatacctgaactggtatcagcagaagcctggtaaggcaccgaagctgctgatctattctgcctccagcctgcaaagcggcgtcccgtcacgctttagtggctctggttctgggaccgacttcactctcaccattagcagtctgcaaccggaggacttcgcgacatactattgccagcagtcttactccgcaccgtatagctttggaggcggtaccaaggtggagatcaaa

SEQ ID NO: 177 - skipped

SEQ ID NO: 178 - protein, 8 aa, synthetic construct
GFSLRNYA

SEQ ID NO: 179 - skipped

SEQ ID NO: 180 - protein, 8 aa, synthetic construct
ISGGGGRT

SEQ ID NO: 181 - skipped

SEQ ID NO: 182 - protein, 17 aa, synthetic construct
ARTGPGIVGRVSGAFDV

SEQ ID NO: 183-191 - skipped (9 entries)

SEQ ID NO: 192 - protein, 6 aa, synthetic construct
QSITKY

SEQ ID NO: 193-195 - skipped (3 entries)

SEQ ID NO: 196 - protein, 9 aa, synthetic construct
QQSYSAPYS

SEQ ID NO: 197-206 - skipped (10 entries)

SEQ ID NO: 207 - protein, 127 aa, synthetic construct
EVQLVESGGGLVQPGGSLRLSCAASGFTFSHYAMSWFRQAPGKGRELVAVIYSGGSTYYPDSVEGRFTISRDNAKKMLYLQMNSLRAEGDTAVYYCARRRSWWSNRDRYHYNAFDYWGQGTLVTVSS

SEQ ID NO: 208 - DNA, 381 bp, synthetic construct
  source 1..381: mol_type = other DNA
gaggttcagttggtcgagtctggtggtgggctcgtgcaacccggaggctccctgaggttgagctgcgccgcttcaggctttacattcagccactacgccatgagctggttcagacaagcacctggtaagggcagagaactggtggctgtgatctactccggaggcagcacctattaccccgattccgtggagggacggtttactatttcaagggacaacgctaagaaaatgctataccttcagatgaatagtttgagggcagagggagatacggccgtgtattactgtgctcgcagacggagctggtggtcaaaccgtgatcgctatcactataacgcctttgactactggggccagggaaccctggtcaccgtctcctca

SEQ ID NO: 209 - skipped

SEQ ID NO: 210 - protein, 8 aa, synthetic construct
GFTFSHYA

SEQ ID NO: 211 - skipped

SEQ ID NO: 212 - protein, 7 aa, synthetic construct
IYSGGST

SEQ ID NO: 213 - skipped

SEQ ID NO: 214 - protein, 20 aa, synthetic construct
ARRRSWWSNRDRYHYNAFDY

SEQ ID NO: 215-224 - skipped (10 entries)

SEQ ID NO: 225 - protein, 127 aa, synthetic construct
EVQLVESGGGLVQPGGSLRLSCAASGFTFSGYAMSWFRQAPGKGRELVAVIYSGGSTYYPDSVEGRFTISRDNAKKMLYLQMNSLRAEGDTAVYYCARRRSWWSNRDRYHYNAFDYWGQGTLVTVSS

SEQ ID NO: 226 - DNA, 381 bp, synthetic construct
  source 1..381: mol_type = other DNA
gaggttcagttggtcgagtctggtggtgggctcgtgcaacccggaggctccctgaggttgagctgcgccgcttcagggttcaccttttccggctacgccatgagctggttcagacaagcacctggtaagggcagagaactggtggctgtgatctactccggaggcagcacctattaccccgattccgtggagggacggtttactatttcaagggacaacgctaagaaaatgctataccttcagatgaatagtttgagggcagagggagatacggccgtgtattactgtgctcgcagacggagctggtggtcaaaccgtgatcgctatcactataacgcctttgactactggggccagggaaccctggtcaccgtctcctca

SEQ ID NO: 227 - skipped

SEQ ID NO: 228 - protein, 8 aa, synthetic construct
GFTFSGYA

SEQ ID NO: 229 - skipped

SEQ ID NO: 230 - protein, 7 aa, synthetic construct
IYSGGST

SEQ ID NO: 231 - skipped

SEQ ID NO: 232 - protein, 20 aa, synthetic construct
ARRRSWWSNRDRYHYNAFDY

SEQ ID NO: 233-242 - skipped (10 entries)

SEQ ID NO: 243 - protein, 119 aa, synthetic construct
EVQLVESGGGLVQPGGSLRLSCAASGFSFSSYLMAWFRQAPGKGRELVAGISRSGGGTYYPDSVEGRFTISRDNSKKMVYLQMNSLRAEGDTAVYYCARDAGGWGMDVWGQGTLVTVSS

SEQ ID NO: 244 - DNA, 357 bp, synthetic construct
  source 1..357: mol_type = other DNA
gaggttcagttggtcgagtctggtggtgggctcgtgcaacccggaggctccctgaggttgagctgcgccgcttcaggcttttctttctccagctacctgatggcctggttcagacaagcacctggtaagggcagagaactggtggctggcatcagcaggtctggcggagggacatactaccccgattccgtggagggacggtttactatttcaagggacaactctaagaaaatggtataccttcagatgaatagtttgagggcagagggagatacggccgtgtattactgtgccagggacgctggcgggtggggcatggatgtttggggccagggaaccctggtcaccgtctcctca

SEQ ID NO: 245 - skipped

SEQ ID NO: 246 - protein, 8 aa, synthetic construct
GFSFSSYL

SEQ ID NO: 247 - skipped

SEQ ID NO: 248 - protein, 8 aa, synthetic construct
ISRSGGGT

SEQ ID NO: 249 - skipped

SEQ ID NO: 250 - protein, 11 aa, synthetic construct
ARDAGGWGMDV

SEQ ID NO: 251-260 - skipped (10 entries)

SEQ ID NO: 261 - protein, 119 aa, synthetic construct
EVQLVESGGGLVQPGGSLRLSCAASGFSFSSYLMAWFRQAPGKGRELVAGISGSGAVTYYPDSVEGRFTISRDNSKKMVYLQMNSLRAEGDTAVYYCARDAGGWGMDVWGQGTLVTVSS

SEQ ID NO: 262 - DNA, 357 bp, synthetic construct
  source 1..357: mol_type = other DNA
gaggttcagttggtcgagtctggtggtgggctcgtgcaacccggaggctccctgaggttgagctgcgccgcttcaggcttttctttctccagctacctgatggcctggttcagacaagcacctggtaagggcagagaactggtggctggcatctccgggagcggagccgtgacctactaccccgattccgtggagggacggtttactatttcaagggacaactctaagaaaatggtataccttcagatgaatagtttgagggcagagggagatacggccgtgtattactgtgccagggacgctggcgggtggggcatggatgtttggggccagggaaccctggtcaccgtctcctca

SEQ ID NO: 263 - skipped

SEQ ID NO: 264 - protein, 8 aa, synthetic construct
GFSFSSYL

SEQ ID NO: 265 - skipped

SEQ ID NO: 266 - protein, 8 aa, synthetic construct
ISGSGAVT

SEQ ID NO: 267 - skipped

SEQ ID NO: 268 - protein, 11 aa, synthetic construct
ARDAGGWGMDV

SEQ ID NO: 269-278 - skipped (10 entries)

SEQ ID NO: 279 - protein, 122 aa, synthetic construct
EVQLVESGGGLVQPGGSLRLSCAASGFTFSNYAMSWFRQAPGKGRELVASITGSGATTFYPDSVEGRFTISRDNAEKLVYLQMNSLRAEGDTAVYYCAKGHGDWYGSGIDIWGQGTLVTVSS

SEQ ID NO: 280 - DNA, 366 bp, synthetic construct
  source 1..366: mol_type = other DNA
gaggttcagttggtcgagtctggtggtgggctcgtgcaacccggaggctccctgaggttgagctgcgccgcttcaggcttcacctttagcaactacgccatgtcctggttcagacaagcacctggtaagggcagagaactggtggcttccatcacagggtctggcgctactaccttctaccccgattccgtggagggacggtttactatttcaagggacaacgctgagaaattggtataccttcagatgaatagtttgagggcagagggagatacggccgtgtattactgtgctaagggtcacggagactggtatggctccggcatcgatatttggggccagggaaccctggtcaccgtctcctca

SEQ ID NO: 281 - skipped

SEQ ID NO: 282 - protein, 8 aa, synthetic construct
GFTFSNYA

SEQ ID NO: 283 - skipped

SEQ ID NO: 284 - protein, 8 aa, synthetic construct
ITGSGATT

SEQ ID NO: 285 - skipped

SEQ ID NO: 286 - protein, 14 aa, synthetic construct
AKGHGDWYGSGIDI

SEQ ID NO: 287-296 - skipped (10 entries)

SEQ ID NO: 297 - protein, 121 aa, synthetic construct
EVQLVESGGGLVQPGGSLRLSCAASGFTFNDYAMSWFRQAPGKGRELVARISGSGGTTNYPDSVEGRFTISRDNPKKLVYLQMNSLRAEGDTAVYYCARERLGYPWTFDVWGQGTLVTVSS

SEQ ID NO: 298 - DNA, 363 bp, synthetic construct
  source 1..363: mol_type = other DNA
gaggttcagttggtcgagtctggtggtgggctcgtgcaacccggaggctccctgaggttgagctgcgccgcttcaggctttaccttcaacgactacgccatgagctggttcagacaagcacctggtaagggcagagaactggtggctaggatctccggcagcggagggacaaccaactaccccgattccgtggagggacggtttactatttcaagggacaaccctaagaaattggtataccttcagatgaatagtttgagggcagagggagatacggccgtgtattactgtgccagggagcggctggggtacccttggaccttcgacgtgtggggccagggaaccctggtcaccgtctcctca

SEQ ID NO: 299 - skipped

SEQ ID NO: 300 - protein, 8 aa, synthetic construct
GFTFNDYA

SEQ ID NO: 301 - skipped

SEQ ID NO: 302 - protein, 8 aa, synthetic construct
ISGSGGTT

SEQ ID NO: 303 - protein, 22 aa, synthetic construct
MGILKLQVFLIVLSVALNHLKA

SEQ ID NO: 304 - protein, 13 aa, synthetic construct
ARERLGYPWTFDV

SEQ ID NO: 305 - protein, 121 aa, synthetic construct
EVQLVESGGGLVKPGGSLKLSCAASGFTFSDYYMFWVRQTPEKRLEWVATISDGGSYTYYPGGVKGRFTISRDNAKNNLYLQMTSLKSEDTAMYFCARDGGMLTTRAMDYWGQGTSVTVSS

SEQ ID NO: 306 - protein, 60 aa, synthetic construct
KCNTATCATQRLANFLVHSSNNFGPILPPTNVGSNTYGSGSATGGSGSGASSGSGSATGS

SEQ ID NO: 307 - protein, 107 aa, synthetic construct
DIQMTQTTSSLSASLGDRVTISCSASQGISNYLNWYQQKPDGTVKLLIYYTSSLHSGVPSRFSGSGSGTDYSLTISNLEPEDIATYYCQHYSEFPFTFGSGTKLEIK

SEQ ID NO: 308 - protein, 507 aa, synthetic construct
KCNTATCATQRLANFLVHSSNNFGPILPPTNVGSNTYGSGSATGGSGSGASSGSGSATGSEVQLVESGGGLVKPGGSLKLSCAASGFTFSDYYMFWVRQTPEKRLEWVATISDGGSYTYYPGGVKGRFTISRDNAKNNLYLQMTSLKSEDTAMYFCARDGGMLTTRAMDYWGQGTSVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLG

SEQ ID NO: 309 - protein, 214 aa, synthetic construct
DIQMTQTTSSLSASLGDRVTISCSASQGISNYLNWYQQKPDGTVKLLIYYTSSLHSGVPSRFSGSGSGTDYSLTISNLEPEDIATYYCQHYSEFPFTFGSGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 310 - DNA, 363 bp, synthetic construct
  source 1..363: mol_type = other DNA
gaggtgcagctggtggaaagcggcggcggcctggtgaagcctggcggcagcctgaagctgagctgcgccgcctccggcttcaccttctctgactactacatgttctgggttaggcagacccccgagaagaggctggagtgggtggccaccatcagcgacggcggcagctacacctactaccccggcggcgttaagggcaggttcaccatcagcagggacaacgccaagaacaacctgtacctgcagatgaccagcctgaaaagcgaggacaccgccatgtacttttgcgccagggacggcggcatgctgaccacacgcgccatggactactggggccagggcaccagcgtgaccgtgagcagc

SEQ ID NO: 311 - DNA, 180 bp, synthetic construct
  source 1..180: mol_type = other DNA
aagtgcaataccgctacctgcgctacccagaggctcgctaacttcctggtgcacagctctaacaatttcggccccatcctgccccccaccaacgtgggaagcaacacctacggaagcggcagtgccaccggcggctctgggagcggcgcctcctctggaagcggctctgccaccggcagc

SEQ ID NO: 312 - DNA, 321 bp, synthetic construct
  source 1..321: mol_type = other DNA
gacatccagatgacccagaccaccagcagcctgagcgcctccctgggcgacagggtgaccatcagctgcagcgccagccagggcatcagcaactacctgaactggtaccagcagaagcccgacggcaccgtgaagctgctgatctactacacctccagcctgcacagcggcgtgcccagcaggttcagcggcagcggaagcggcaccgactacagcctgaccatcagcaacctcgaacccgaggacatcgccacctactactgccagcactacagcgagttccccttcaccttcggcagcggcaccaagctggagatcaag

SEQ ID NO: 313 - DNA, 1521 bp, synthetic construct
  source 1..1521: mol_type = other DNA
aagtgcaataccgctacctgcgctacccagaggctcgctaacttcctggtgcacagctctaacaatttcggccccatcctgccccccaccaacgtgggaagcaacacctacggaagcggcagtgccaccggcggctctgggagcggcgcctcctctggaagcggctctgccaccggcagcgaggtgcagctggtggaaagcggcggcggcctggtgaagcctggcggcagcctgaagctgagctgcgccgcctccggcttcaccttctctgactactacatgttctgggttaggcagacccccgagaagaggctggagtgggtggccaccatcagcgacggcggcagctacacctactaccccggcggcgttaagggcaggttcaccatcagcagggacaacgccaagaacaacctgtacctgcagatgaccagcctgaaaagcgaggacaccgccatgtacttttgcgccagggacggcggcatgctgaccacacgcgccatggactactggggccagggcaccagcgtgaccgtgagcagcgcatccaccaagggcccatccgtcttccccctggcgccctgctccaggagcacctccgagagcacagccgccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacgaagacctacacctgcaacgtagatcacaagcccagcaacaccaaggtggacaagagagttgagtccaaatatggtcccccatgcccaccttgcccagcacctgagttcctggggggaccatcagtcttcctgttccccccaaaacccaaggacactctcatgatctcccggacccctgaggtcacgtgcgtggtggtggacgtgagccaggaagaccccgaggtccagttcaactggtacgtggatggcgtggaggtgcataatgccaagacaaagccgcgggaggagcagttcaacagcacgtaccgtgtggtcagcgtcctcaccgtcctgcaccaggactggctgaacggcaaggagtacaagtgcaaggtgtccaacaaaggcctcccgtcctccatcgagaaaaccatctccaaagccaaagggcagccccgagagccacaggtgtacaccctgcccccatcccaggaggagatgaccaagaaccaggtcagcctgacctgcctggtcaaaggcttctaccccagcgacatcgccgtggagtgggagagcaatgggcagccggagaacaactacaagaccacgcctcccgtgctggactccgacggctccttcttcctctacagcaggctaaccgtggacaagagcaggtggcaggaggggaatgtcttctcatgctccgtgatgcatgaggctctgcacaaccactacacacagaagagcctctccctgtctctgggt

SEQ ID NO: 314 - DNA, 642 bp, synthetic construct
  source 1..642: mol_type = other DNA
gacatccagatgacccagaccaccagcagcctgagcgcctccctgggcgacagggtgaccatcagctgcagcgccagccagggcatcagcaactacctgaactggtaccagcagaagcccgacggcaccgtgaagctgctgatctactacacctccagcctgcacagcggcgtgcccagcaggttcagcggcagcggaagcggcaccgactacagcctgaccatcagcaacctcgaacccgaggacatcgccacctactactgccagcactacagcgagttccccttcaccttcggcagcggcaccaagctggagatcaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 315 - protein, 5 aa, synthetic construct
DYYMF

SEQ ID NO: 316 - protein, 17 aa, synthetic construct
TISDGGSYTYYPGGVKG

SEQ ID NO: 317 - protein, 12 aa, synthetic construct
DGGMLTTRAMDY

SEQ ID NO: 318 - protein, 11 aa, synthetic construct
SASQGISNYLN

SEQ ID NO: 319 - protein, 7 aa, synthetic construct
YTSSLHS

SEQ ID NO: 320 - protein, 9 aa, synthetic construct
QHYSEFPFT

SEQ ID NO: 321 - protein, 67 aa, synthetic construct
TPIESHQVEKRKCNTATCATQRLANFLVHSSNNFGAILSSTNVGSNTYGKRNAVEVLKREPLNYLPL

SEQ ID NO: 322 - protein, 37 aa, synthetic construct [sequence omitted]